(19) United States
(12) Patent Application Publication
(10) Pub. No.: US 2012/0245553 A1
(43) Pub. Date: Sep. 27, 2012

(54) INTRAGASTRIC VOLUME OCCUPYING DEVICE WITH ACTIVE AGENTS

(75) Inventors: Joseph S. Raven, Goleta, CA (US); Janel A. Birk, Oxnard, CA (US)

(73) Assignee: ALLERGAN, INC., Irvine, CA (US)

(21) Appl. No.: 13/072,594

(22) Filed: Mar. 25, 2011

(51) Int. Cl. A61M 31/00; A61M 29/00; A61N 1/30

(52) U.S. Cl. 604/500; 606/192; 604/19; 604/98.01

(57) ABSTRACT

An implantable intragastric volume occupying device system is provided which generally includes a volume occupying device and an active agent, for example, a metabolic agent or satiety inducing agent. The volume occupying device may be structured to contain the agent and permit controlled release of the agent to the patient while the volume occupying device is positioned within the patient's stomach. Methods for treating obesity are also provided which include positioning a volume occupying device in the stomach of a patient and administering a satiety inducing agent to the patient while the volume occupying device is positioned in the stomach. In one embodiment, the active agent may be contained in a reservoir and dispensed to a portion of the patient's body.

### FIELD

The present invention relates to intragastric volume occupying devices used for treatment of obesity and obesity related disorders, and more specifically relates to an intragastric volume occupying device system including active agents.

### BACKGROUND

Intragastric volume occupying devices have a successful history of inducing weight loss in obese patients. The volume occupying device is positioned within the stomach and occupies space within the stomach. Generally, this reduces the effective interior volume of the stomach, which allows the patient to ingest only a small amount of food before the patient begins to feel satiated and full. Further, the volume occupying device activates mechanoreceptors of the stomach that are known to produce satiety, which will be engaged sooner than with a standard meal. Consequently, the patient is less likely to eat to excess. With reduced caloric intake, the patient loses weight. It is known that some patients, however, reach a “plateau” in their rate of weight loss over time, even with the volume occupying device in place.

Despite the relative safety and success of intragastric volume occupying device therapy in treating obesity and obesity related conditions, there remains a need for improved systems and methods for treating obesity and obesity related conditions in some patients.

### SUMMARY OF THE INVENTION

The present invention provides an intragastric volume occupying device system generally comprising an intragastric volume occupying device configured to be placed in the stomach of a patient. Further, in embodiments of the present invention, the intragastric volume occupying device is capable of dispensing an active agent, such as but not limited to, a metabolic agent, for example, a satiety inducing agent, to the patient while the intragastric volume occupying device is positioned in the patient's stomach. The system may provide more effective obesity treatment relative to obesity treatment using an intragastric volume occupying device alone.

In one embodiment, the present invention comprises an intragastric volume occupying device including a reservoir configured to contain an active agent being effective, when released into the patient, to at least assist in effecting weight loss in the patient. The reservoir has an outlet configured to allow the active agent to exit the reservoir and contact a portion of the patient's body. A tube may be coupled to the reservoir to allow the active agent to be distributed to a desired portion of the patient's body. In one embodiment, at least a portion of the volume occupying device includes a semi-permeable membrane that is configured to allow the active agent to exit the reservoir and contact a portion of the patient's body.

In some embodiments of the invention, the satiety inducing agent is a hormone, for example a peptide hormone. The peptide hormone may be at least one agent such as Glucagon-like peptide (GLP-1), Oxyntomodulin (OXM), Peptide YY (PYY), Pancreatic Polypeptide (PP), Insulin, Leptin, Gastrin, Ghrelin blocker, inhibitors of DPP-IV, Amylin, and combinations thereof. The satiety inducing agent may be Cholecystokinin (CCK), which may suppress appetite when administered with or without gastric distension.

It is to be appreciated that the active agents useful in the present invention are not limited to satiety inducing agents but may also include any active agents, for example, other metabolic agents, that may provide some benefit to a patient suffering from obesity and/or obesity related conditions.

In one embodiment, the intragastric volume occupying device may be configured as an inflatable balloon. Designs of intragastric volume occupying devices configured as inflatable balloons are discussed in U.S. patent application Ser. No. 12/698,906, titled “Remote Deflation of Intragastric Balloon,” filed Feb. 2, 2010, the entire disclosure of which is incorporated herein by reference.

In one embodiment, the present invention comprises a method for the treatment of obesity, comprising the step of implanting a volume occupying device into a patient's stomach. The volume occupying device is configured to include a reservoir, configured to contain an active agent being effective, when released into the patient, to at least assist in effecting weight loss in the patient. In one embodiment, at least a portion of the volume occupying device includes a semi-permeable membrane that is configured to allow the active agent to exit the reservoir and contact a portion of the patient's body.

In one embodiment, the present invention comprises a method for the treatment of obesity comprising the steps of inserting an electrode into a patient's body laparoscopically and coupling the electrode to the lower third of the patient's esophagus. The electrode is configured to apply electric stimulation to the lower third of the patient's esophagus. The electrode is utilized in combination with a volume occupying device positioned within the patient's stomach.

In one embodiment, an ancillary device is incorporated into the volume occupying device and the ancillary device includes, or is capable of dispensing to the patient, a satiety inducing agent. The ancillary device may be structured to provide controlled release of the satiety inducing agent to the patient.

For example, the ancillary device may comprise a membrane or film permeable to a satiety inducing agent. The agent may be covered or enclosed by the membrane and is released into the body by diffusion through the membrane.

In one embodiment, the ancillary device may comprise a composition including a matrix material and a satiety inducing agent combined with the matrix material. The matrix material may be a biodegradable or bioerodible material, for example, a bioerodible polymer which, during erosion thereof in the body, releases the agent from the composition in a controlled manner.

In one embodiment, the ancillary device may be a non-bioerodible material. The device may include structures for containing and releasing the satiety inducing agents, for example, in a controlled manner. In one embodiment, the ancillary device includes recessions, pores or grooves capable of containing a satiety inducing agent.

In one embodiment, the ancillary device further includes a film or membrane in contact with the agent and capable of releasing the agent from the ancillary device and into the patient, for example, at a controlled rate.

In one embodiment, the volume occupying device itself is structured to be capable of releasing a satiety inducing agent into the patient at a controlled rate.

In one embodiment, the present invention comprises an intragastric volume occupying device system for the treatment of obesity, comprising a volume occupying device configured to occupy volume within a patient's stomach, an implantable sensor coupled to the volume occupying device, and configured to sense a biological characteristic of the patient, and an external control device configured to receive a telemetric signal sent in response to a biological characteristic sensed by the sensor, and to produce a notification in response to the signal to perform an action effective to vary the biological characteristic sensed by the sensor. The biological characteristic may comprise a hormone level of the patient. The action may comprise injection of an active agent into the patient's body, inhaling of an active agent by the patient, drinking of an active agent by the patient, application of a patch to the patient's body being capable of distributing an active agent to the patient, spraying of an active agent into the patient's mouth, swallowing of a pill by the patient containing an active agent, insertion of a gum or a film containing an active agent into the patient's mouth. A combination of actions may be taken, in response to the biological characteristic sensed by the sensor.

Each and every feature described herein, and each and every combination of two or more of such features, is included within the scope of the present invention provided that the features included in such a combination are not mutually inconsistent.

### BRIEF DESCRIPTION OF THE DRAWINGS

FIG. 1 is a perspective view of a system for the treatment of obesity, according to one or more embodiments of the present invention.

FIG. 2 is a side cross-sectional view of an intragastric volume occupying device, according to one or more embodiments of the present invention.

FIG. 3 is a side cross-sectional view of an intragastric volume occupying device, according to one or more embodiments of the present invention.

FIG. 4 is a perspective view of an intragastric volume occupying device, according to one or more embodiments of the present invention.

FIG. 5 is a side cross-sectional view of an intragastric volume occupying device, according to one or more embodiments of the present invention.

FIG. 6 is a side cross-sectional view of an intragastric volume occupying device, according to one or more embodiments of the present invention.

FIG. 7 is a side cross-sectional view of an intragastric volume occupying device, according to one or more embodiments of the present invention.

FIG. 8 is a schematic view of a reservoir, according to one or more embodiments of the present invention.

FIG. 9 is a schematic view of a reservoir, according to one or more embodiments of the present invention.

FIG. 10 is a flowchart representing an exemplary method for the treatment of obesity, according to one or more embodiments of the present invention.

FIG. 11 is a perspective view of an intragastric volume occupying device, according to one or more embodiments of the present invention.

FIG. 12 is a perspective view of a system for the treatment of obesity, according to one or more embodiments of the present invention.

FIG. 13 is a flowchart representing an exemplary method for the treatment of obesity, according to one or more embodiments of the present invention.

FIG. 14 is a perspective view of an intragastric volume occupying device, according to one or more embodiments of the present invention.

FIGS. 15A and 15B are perspective views of surface structures useful for containing active agents in conjunction with an intragastric volume occupying device, according to one or more embodiments of the present invention.

FIG. 16 is a simplified representation of a diffusion material useful for controlling release of active agents in conjunction with an intragastric volume occupying device, according to one or more embodiments of the present invention.

FIG. 17 is a perspective view of a system for the treatment of obesity, according to one or more embodiments of the present invention.

### DETAILED DESCRIPTION

The present invention provides for intragastric volume occupying device systems for the treatment of obesity, including intragastric volume occupying devices, or volume occupying devices capable of occupying volume within a patient's stomach. In embodiments of the present invention, the intragastric volume occupying device system may be capable of distributing an active agent to a portion of a patient's body. The active agent may be effective, when released into the patient, to at least assist in effecting weight loss in the patient. An embodiment of the intragastric volume occupying device system that includes an active agent, may provide more effective obesity treatment relative to obesity treatment using an intragastric volume occupying device alone.

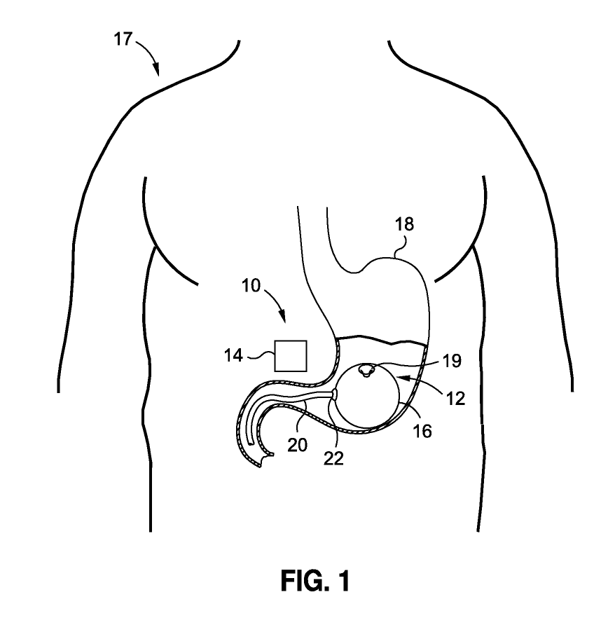

FIG. 1 illustrates an embodiment of an intragastric volume occupying device system 10 including an intragastric volume occupying device 12 (equivalently referred to as an “IVOD”) and a sensor 14. The sensor 14 could be located separately from the intragastric volume occupying device 12 or could be integrated into the intragastric volume occupying device 12, depending on the parameter that will be sensed. The intragastric volume occupying device 12 is configured to occupy volume within the patient's stomach 18. The intragastric volume occupying device 12 includes a shell 16 configured to be positioned within a patient's 17 stomach 18. A fill valve 19 is coupled to the shell 16. A tube 20 is coupled to the shell 16. An outlet device 22 is coupled to the shell 16. The outlet device 22 connects to the tube 20.

The size, or volume, of the intragastric volume occupying device 12 is variable, and may be selected as desired, to increase or decrease the size of the intragastric volume occupying device 12, in conjunction with the active agent therapy in any of the embodiments of this application.

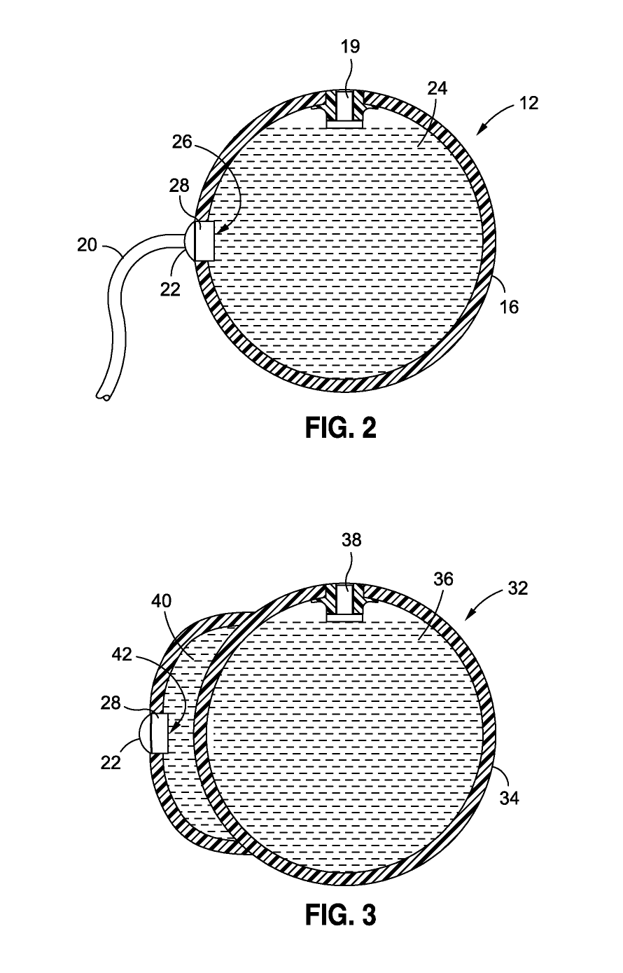

Referring to FIG. 2, a reservoir 24 may be positioned within the interior of the intragastric volume occupying device 12. The shell 16 forms an outer surface of the reservoir 24. The reservoir 24 may be configured to contain an active agent, being effective, when released into the patient 17, to at least assist in effecting weight loss in the patient 17. The active agent may be a metabolic agent, for example, a satiety inducing agent, or for example, a satiety gut hormone or bioactive molecule. Although the present disclosure will typically be discussing, specifically, satiety inducing agents, it is to be appreciated that the present invention, in all embodiments, is not limited to active agents that are, specifically, satiety inducing agents. Active agents useful with the present invention are intended to include other compositions, drugs or other agents, for example, agents that affect body metabolism without necessarily affecting satiety, that are believed to be effective, at least to some degree, in facilitating weight loss in a human being.

FIG. 2 illustrates a cross-section view of the intragastric volume occupying device 12 shown in FIG. 1. The shell 16 of the volume occupying device 12 comprises an inflatable balloon structure that may be filled with the active agent in a fluid form. The shell 16 may have a substantially spherical shape, and may define the hollow cavity that forms the reservoir 24. The shell 16 may be made from a flexible material that allows the shell 16 to deform, and vary in size. For example, the shell 16 may be made from silicone or other equivalent materials. The shell 16 may deform based on the amount of fluid contained in the reservoir 24. For example, the shell 16 may increase in interior volume when fluid enters the reservoir 24. In one embodiment, the shell 16 may be made from an elastic material that stretches when fluid enters the reservoir 24.

The fill valve 19 comprises a fluid entry channel that allows the active agent to fill the reservoir 24. The fill valve 19 is positioned on the shell 16 in an orientation in which a physician may access the fill valve 19 to insert the active agent into the reservoir 24 and inflate the intragastric volume occupying device 12. The fill valve 19 may comprise a one-way valve, or a check valve, that does not permit the active agent to exit the reservoir 24 once it has entered the reservoir 24. The one-way valve may be configured as a leaf-valve, a duckbill valve, a diaphragm valve, or the like.

The shell 16 includes an outlet 26 that allows the active agent to exit the reservoir 24. The outlet 26 of the reservoir 24 may comprise a fluid channel or passageway that allows the active agent to pass from the reservoir 24 to the exterior of the intragastric volume occupying device 12, to contact a portion of the patient's body.

The tube 20 may couple to the outlet 26 of the reservoir 24. The tube 20 may comprise a tubular member, or fluid conduit, made, for example, from silicone, or the like. One end of the tube 20 may couple to the outlet 26 of the reservoir 24. The other end of the tube 20 may be an open end, and may be positioned in a desired position within the patient's body, to allow the active agent to flow from the reservoir 24 and distribute to the desired portion of the patient's body. As shown in FIG. 1, for example, one end of the tube 20 extends into a portion of the upper intestines of the patient's 17 body. The active agent may flow from the reservoir 24, through the tube 20, and be absorbed by the tissues of the intestines. In one embodiment, the tube 20 may be fixed to any desired portion of the patient's body using sutures, tacks, adhesives, or the like.

Referring back to FIG. 2, the outlet 26 may include an outlet device 22. The outlet device 22 may allow, enhance, prevent, or impede the ability of the active agent to exit from the reservoir 24. The outlet device 22 may be coupled to the shell 16, in a position near a surface of the intragastric volume occupying device 12. The outlet device 22 may comprise a device such as a pump or a valve, or may comprise a combination of a pump and a valve. The pump and/or valve may be powered inductively from a remote device and/or through a battery (not shown) that may be charged prior to implantation of the intragastric volume occupying device 12, or may be charged inductively after implantation, through appropriate means.

The pump may comprise a micro-pump, for example, a piezoelectric pump capable of driving fluid through use of a diaphragm mechanism. In addition, the pump may comprise any other desired type of implantable pump or micro-pump, capable of providing equivalent operation.

The valve may comprise a piezoelectric valve, for example a valve capable of allowing fluid to pass through the outlet 26 with a powered diaphragm mechanism. The valve may also comprise any other desired type of valve device or micro-valve capable of providing equivalent operation. Embodiments of pumps and/or valves that may be preferably utilized in the present invention are disclosed and discussed in U.S. patent application Ser. No. 12/428,311, titled “Remotely Adjustable Gastric Banding System,” filed on Apr. 22, 2009, the entire disclosure of which is incorporated herein by reference.

The embodiments of the outlet device 22 that include powered mechanisms (e.g., the pump and the valve) may be used in conjunction with a controller 28. The controller 28 may be coupled to the shell 16, near the outlet device 22. The controller 28 may include an appropriate fluid conduit that allows the active agent to pass from the reservoir 24, through the outlet 26. The controller 28 may comprise circuitry and/or a power system capable of operating the outlet device 22 and communicating with other devices utilized in the intragastric volume occupying device system 10. The controller 28 may include transmitter and receiver devices, which may send and receive signals telemetrically.

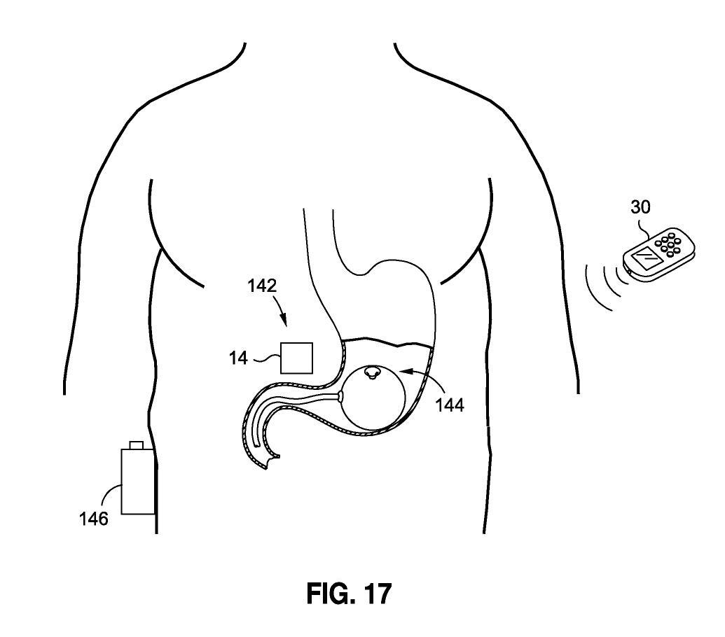

The controller 28 may be capable of causing the outlet device 22 to either increase a flow of active agent from the reservoir 24, or to decrease a flow of active agent from the reservoir 24, in response to signals sent by either the sensor 14 (shown in FIG. 1) or an external controller device 30 (discussed in relation to FIG. 17). For example, the controller 28 may cause an embodiment of the outlet device 22 comprising a pump, to pump active agent from the reservoir 24 in response to a signal sent from the sensor 14. In addition, the controller 28 may be configured to open or close an embodiment of the outlet device 22 comprising a powered valve, in response to a signal sent from the sensor 14.

The receiver of the controller 28 may include an antenna, capable of receiving signals transmitted from either inside the body or outside the body. If signals are transmitted from inside the body, the signals may be sent from the sensor 14 (shown in FIG. 1), which may be configured to wirelessly transmit signals to the controller 28. If signals are transmitted from outside the body, the signals may be sent from an external control device 30 (discussed in relation to FIG. 17). Both the sensor 14 and the external control device 30 may be capable of transmitting signals to the controller 28. The transmitted signals may cause the controller 28 to increase or decrease the rate the active agent exits the reservoir 24. For example, the transmitted signals may instruct the outlet device 22 to either pump an active agent through the outlet 26, or pump less of the active agent through the outlet 26, or to open or close a powered valve incorporated with the outlet device 22. The controller 28 may be powered by similar means as the outlet device 22, namely, through battery power or through induction.

The transmitter of the controller 28 may include an antenna, which may be the same antenna as used with the receiver, and is capable of transmitting signals outside the body. The transmitted signals may be utilized in various embodiments of the intragastric volume occupying system 10. For example, in one embodiment, the controller 28 may include a flow meter, and the transmitter may be capable of sending a signal to a physician, indicating whether a certain amount of active agent has passed through the outlet 26. In one embodiment, the controller 28 may detect whether the pump is operating, or the valve is open. In this embodiment, the transmitter may alert a physician when the active agent is being distributed from the reservoir 24.

In one embodiment, a pressure sensor may be incorporated with the reservoir 24, capable of signaling to the controller 28 when the pressure level of the reservoir 24 is low enough to require more active agent to be inserted into the reservoir 24, or to require the intragastric volume occupying device 12 to be replaced. The transmitter may send a signal to an external control device 30 (discussed in relation to FIG. 17), indicating a fluid level or volume of the reservoir 24 to the user of the external control device 30. In one embodiment, the controller 28 may include a processor and a memory, the memory being capable of storing instructions executable by the processor. The instructions may be preprogrammed into the controller 28 prior to implantation of the intragastric volume occupying device, or may be received by the receiver of the controller 28, and set into memory by the patient or physician wirelessly, after implantation. The instructions may produce any of the actions performed by the controller 28.

Referring back to FIG. 1, the system 10 may further include a biological sensor 14 which may be utilized in combination with the reservoir 24 shown in FIG. 2. The sensor 14 may comprise circuitry including a biological sensor capable of detecting a desired biological characteristic, property, or value. The biological characteristic may be a hormone level, which may be detected through means known to those skilled in the art. For example, a hormone level of the patient may be measured by measuring the dielectric constant of interstitial fluid, intra-peritoneal fluid, or blood plasma, across two electrodes, in a manner that reflects the hormone concentration of the patient. The sensor 14 may include a receiver and a transmitter, which are respectively capable of receiving and sending signals either to the controller 28 of the reservoir 24, or to a receiver located exterior to the patient's body.

The sensor 14 may be configured to cause a signal to be sent to the controller 28 (shown in FIG. 2) or an external control device 30 (shown in FIG. 17), in response to the measured biological characteristic of the patient. For example, the sensor 14 may be configured to store a threshold detection level for a biological characteristic within a patient's body. If the detected biological characteristic decreases below the threshold value, then the sensor 14 may be configured to send a signal to the controller 28 of the reservoir 24 or to the external control device 30. The signal received by the controller 28 may cause the controller 28 to instruct, control, or power, the outlet device 22 to vary a rate the active agent is dispensed from the reservoir 24 and delivered to a portion of the patient's body (e.g. the part of the patient's body where the output of the tube 20 is located). In this embodiment, the signal will preferably increase the rate the active agent is dispensed from the reservoir 24. In addition, or alternatively, a threshold detection level may be stored or set in the sensor 14 that represents an upper limit of a detected biological characteristic. For example, if the sensor 14 detects the biological characteristic is above a threshold level, then the sensor 14 may send a signal to the controller 28 to instruct, control, or power, the outlet device 22 to reduce the amount of active agent being dispensed from the reservoir 24. The sensor 14 and the reservoir 24 may thus act in a closed feedback loop, which allows the amount of fluid dispensed from the reservoir 24 to be controlled, at least in part, by a biological characteristic of the patient's body. If the biological characteristic is a hormone level, the sensor 14 and the reservoir 24 may then act in a feedback loop to control the hormone level of the patient. The sensor 14 may be powered inductively from a remote device and/or through a battery (not shown) that may be charged prior to implantation of the intragastric volume occupying device system 10, or charged inductively after implantation, through appropriate means.

In one embodiment, the sensor 14 may include a processor and a memory. The processor may be capable of executing instructions stored in the memory. The instructions may be preprogrammed into the sensor 14 prior to implantation, or may be received by the sensor and set into memory by the patient or physician wirelessly, after implantation. The instructions may comprise any of the actions and responses performed by the sensor 14. For example, the instructions may include the threshold detection values set to be detected by the sensor 14.

The active agent that is dispensed from the reservoir 24 may comprise an active agent that is a satiety inducing agent. The satiety inducing agent may be a hormone, for example, a peptide hormone. The hormone may comprise at least one agent such as Glucagon-like peptide (GLP-1), Oxyntomodulin (OXM), Peptide YY (PYY) and Peptide YY (3-36) (PYY (3-36)), Pancreatic Polypeptide (PP), Insulin, Leptin, Gastrin, Ghrelin blocker, inhibitors of DPP-IV, Amylin, and combinations thereof. In addition, the satiety inducing agent may be Cholecystokinin (CCK) or Cholecystokinin 8 (CCK-8) or Pro-opiomelanocortin (POMC), or others, or any combination of the above.

The active agent may also be an agent selected from a list of agents such as Glial-Derived Neurotrophic Factor (GDNF); Serotonin; Dopamine and its Analogues such as: Ibogaine, Noribogaine, 18-MC, and Cabergoline; Ciliary-derived Neurotrophic Factor (CNTF); Cocaine-Amphetamine Regulated Transcript (CART); Serotonin and its Analogues; Gastric Inhibitory Peptide or Glucose-dependant Insulinotropic Peptide (GIP); Neuropeptide Y (NPY) receptor antagonists and iRNA/siRNA; Orexin A and B receptor antagonists and iRNA/siRNA; Agouti Related Peptide (AgRP) receptor antagonists and iRNA/siRNA; Cannabanoid receptor antagonists and iRNA/siRNA; the Melanocortins: Pro-Opiomelanocortin (POMC), Alpha and Beta Melanocyte Stimulating Hormone (α and β MSH); Melanin Concentrating Hormone (MCH) receptor antagonists and iRNA/siRNA; Adenosine Mono-Phosphate activated protein Kinase (AMPK); 5-aminoimidazole-4-carboxamide-1-β-D-ribofuranoside (AICAR); and Peroxisome Proliferator-Activated Receptor Delta Agonist (PPAR5-agonist), or others, or any combinations of the above.

Discussions of gastrointestinal hormones that control appetite can be found in Chaudhri. O. B., Wynne, K., and Bloom, S. R. 2008. “Can Gut Hormones Control Appetite and Prevent Obesity?”. Diabetes Care 31 (Suppl. 2): s284-s289. Additional information is found in Cummings, David E. and Overduin, J. 2007. “Gastrointestinal Regulation of Food Intake.” J. Clin. Invest. 117: 13-23, the entire disclosures of which are incorporated herein by reference. In the publication, “Can Gut Hormones Control Appetite and Prevent Obesity?” by Chaudhri, et al, research conducted on Gherlin, GLP-1, Oxyntomodulin, Inhibitors of DPP-IV, Amylin, Peptide YY, and Pancreatic Polypeptide to control appetite, are described. These as well as other hormones may be useful in accordance with the present invention. Similarly, “Gastrointestinal Regulation of Food Intake” by David E. Cummings et al describes the efficacy of satiety hormones to boost weight loss.

In other embodiments of the invention, the active agent may be any suitable active agent that will improve the weight-loss effect of the intragastric volume occupying device. For example, the active agent may be an agent that affects metabolism of a patient independently of the effect, if any, on satiety of the patient. Metabolic agents that are known or suspected to have a positive effect on weight loss are known to those of skill in the art.

In one embodiment, the active agent may be contained within microspheres, that are held within the reservoir 24, or any other embodiment of a reservoir discussed throughout this application. The microspheres would be held in solution within the reservoir. The microspheres may be dispensed into the patient's body, to release the active agent contained within the microspheres.

Any of the active agents discussed throughout this application may be bioengineered to resist the breakdown of the active agent. For example, in an embodiment in which the active agent comprises a hormone, enzymes within a patient's body will begin a process of breaking down and rendering the hormone ineffective after the hormone is introduced into the patient's body. The enzymes target specific sites, particularly amino acids of the hormone, to cleave the hormone molecule, thus inactivating the hormone by changing its ability to bind to its receptor or exert its intended effect. To prevent this undesirable result, specific DNA capable of producing the hormone may be identified and modified to reduce the enzymatic degradation. For example, once a specific DNA sequence has been isolated and identified for the hormone of interest, small changes can be made to the DNA coding sequence. By altering the amino acid that is expressed following post-translational processing, insignificant changes can be made to the hormone molecule's stereo-structure while making the hormone relatively resistant to enzymatic degradation, thereby extending its half-life and efficacy. Methods of producing recombinant DNA are discussed in “AN INTRODUCTION TO GENETIC ANALYSIS” by Anthony Griffiths, Jeffery Miller, David Suzuki, Richard Lewontin, and William Gelbert, the entirety of which is incorporated by reference. Further information may also be found in “MOLECULAR CELL BIOLOGY” by Harvey Lodish, Arnold Berk, Paul Matsudaira, Chris Kaiser, Monty Krieger, Matthew Scott, S. Lawrence Zipursky, and James Darnell, the entirety of which is incorporated herein by reference.

In one embodiment, a Phenylethylene glycol (PEG) group may be added to any of the active agents discussed throughout this application, to enhance the effectiveness and longevity of the agent.

Referring to FIG. 1, the intragastric volume occupying device 12 is implanted into the patient's 17 stomach 18 endoluminally. The intragastric volume occupying device 12 is passed through the patient's 17 esophagus in a deflated state, in which no active agent, or a minimal amount of active agent is present in the reservoir 24 (shown in FIG. 2). The intragastric volume occupying device 12 is placed in the desired position within the patient's 17 stomach 18. After the device 12 is in position, a filling mechanism (not shown) then engages the fill valve 19 and fills the reservoir 24 with a desired volume of active agent. The filling mechanism may then be withdrawn from the patient's 17 stomach 18. In an embodiment in which the intragastric volume occupying device 12 includes a tube 20, the tube 20 may be positioned in a desired position within the patient's 17 body. For example, one end of the tube 20 may be positioned in the patient's 17 upper intestines. The tube 20 may be positioned through appropriate endoluminal means.

The sensor 14 of the intragastric volume occupying device system 10 may be positioned laparoscopically within the patient's body in a desired position. Such positions may include locations in which the sensor 14 may detect interstitial fluid, intra-peritoneal fluid, or blood plasma, across two electrodes, in a manner that reflects the hormone concentration of the patient. For example, the sensor 14 may be positioned near highly vascularized and permeable tissue such as a mucous or serous membrane within the patient's body. In one embodiment, the sensor 14 may be positioned endoluminally within the patient's body. In this embodiment, the sensor 14 may be placed within the patient's stomach, or gastrointestinal tract. In one embodiment, the sensor 14 may be integrated as a component of the intragastric volume occupying device 12. In this embodiment, the sensor 14 may be coupled to the shell 16 of the device 12, and positioned along the exterior surface of the shell 16 to measure a local biological characteristic.

Once the intragastric volume occupying device 12 is positioned within the patient's 17 stomach 18, the reservoir 24 (shown in FIG. 2) acts to dispense the active agent to the patient's 17 body, and, as shown in FIG. 1, does so in combination with an intragastric volume occupying device 12 configured to occupy volume within the patient's 17 stomach 18. The use of the reservoir 24 thus serves to enhance the therapeutic properties of the intragastric volume occupying device 12 treatment by distributing an active agent that promotes satiety signals of the patient, or alters the metabolism of the patient, which will cause the patient to lose weight.

The dosage of the active agent that is distributed by the reservoir 24 may be adjusted by a physician to accommodate various personal properties of the patient (e.g., weight loss goal, size of the patient, results of the intragastric volume occupying device and active agent treatment). For example, a physician may communicate with the sensor 14 to set a certain biological threshold detection level (e.g., a hormone threshold detection level), which corresponds to the personal properties of the patient. The sensor 14 may send a signal to the controller 28 or an external control device 30 if a measured biological characteristic deviates from the threshold detection level. In addition, a physician may communicate with the reservoir 24, via the controller 28, to set a degree of flow and volume from the reservoir 24, or to set a pumping or flow rate of the outlet device 22, according to the personal properties of the patient. In addition, the physician may program in the controller 28 a schedule at which the active agent is dispensed from the reservoir 24.

In one embodiment, the output device 22 comprises a check valve, or a one-way valve, that is configured to allow an active agent to flow from the reservoir 24 in response to a force being exerted against the reservoir 24, which may be a force exerted by the patient's 17 stomach 18 or by food within the patient's 17 stomach 18. The patient's 17 stomach 18 may exert a force against the reservoir 24 during activities involving digestion. In addition, food within the patient's 17 stomach 18 may exert a force against the reservoir 24 after it has entered the stomach 18 following its consumption. The force exerted against the reservoir 24 may pressurize the reservoir 24 to a degree that the one-way valve opens, and allows the active agent to exit the reservoir 24. The one-way valve may thus have a set pressure threshold, at, or above which, the one-way valve opens and allows fluid to pass through. The one-way valve may then be capable of beneficially distributing the active agent during times when the patient is eating (e.g., the stomach 18 exerts forces against the reservoir 24, or food entering the stomach 18 exerts forces against the reservoir 24). The active agent may then be dispensed at a time when increased satiety signals would be more useful to reduce the volume of food consumed by the patient, namely, at the eating times of the patient. In one embodiment, the one-way valve may also be a variable one-way valve, or a one-way valve that is capable of varying the pressure the valve opens in response to. The opening pressure of the one-way valve may be varied by the physician mechanically, prior to implantation of the intragastric volume occupying device 12, or mechanically after implantation of the intragastric volume occupying device 12. In one embodiment, the one-way valve may operate in combination with the controller 28, and a physician may communicate wirelessly with the controller 28, to adjust the threshold pressure of the one-way valve.

A possible drawback to the embodiment shown in FIG. 2 is that as the active agent exits the reservoir 24, then the size of the intragastric volume occupying device 12 will decrease. The flexible shell 16 may deflate as the active agent exits the reservoir 24. Thus, the effectiveness of the intragastric volume occupying device 12, to occupy volume within the patient's stomach, will decrease over time, as the volume of active agent in the reservoir 24 decreases. A separate fill reservoir and active agent reservoir may remedy the problem of the size of the reservoir 24 decreasing.

The embodiment of the intragastric volume occupying device 32 shown in FIG. 3 utilizes a separate fill reservoir 36 and an active agent reservoir 40. The intragastric volume occupying device 32 includes a shell 34, which forms a shell around the fill reservoir 36, and an active agent reservoir 40. A fill valve 38, which may be configured similarly as the fill valve 19 shown in FIG. 2, allows fluid to enter and fill the fill reservoir 36. The active agent reservoir 40 is positioned exterior to the fill reservoir 36 and includes an outlet 42 for allowing the active agent to exit the active agent reservoir 40. Thus, fluid does not exit the fill reservoir 36 as the active agent exits from the outlet 42 of the active agent reservoir 40. The size of fill reservoir 36 will not decrease as the active agent exits from the active agent reservoir 40, thus substantially maintaining the size and effectiveness of the intragastric volume occupying device 32 during the obesity treatment.

The active agent reservoir 40 may be filled by a separate filling mechanism, or a filling mechanism integrated with the filling mechanism used with the fill valve 38.

The outlet device 22 and the controller 28 shown in FIG. 3 may be configured similarly as the outlet device 22 and the controller 28 shown in FIG. 2.

In the embodiment shown in FIG. 3, the intragastric volume occupying device 32 does not include a tube, such as a tube 20 shown in FIG. 2, leading from the active agent reservoir 40 to a portion of the patient's body. In this embodiment, the active agent may pass from the active agent reservoir 40 directly to the patient's stomach. A tube may or may not be utilized throughout all embodiments of the intragastric volume occupying device 32 shown throughout this disclosure.

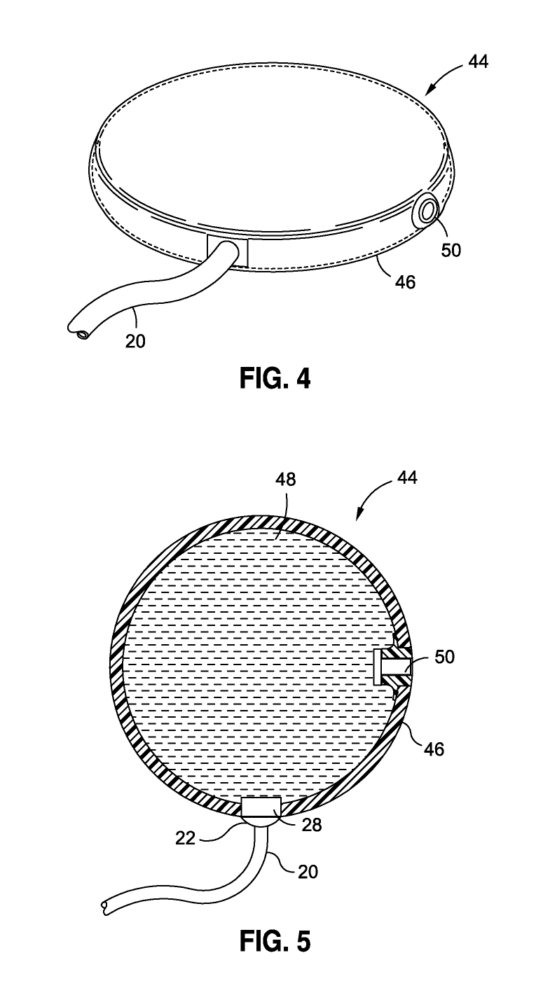

FIG. 4 illustrates an embodiment of an intragastric volume occupying device 44 having a flattened disk-like shape. A shell 46 forming the intragastric volume occupying device 44 is configured as a flexible disk having an upper surface and a lower surface that are substantially flat and parallel to each other. The shell 46 surrounds a reservoir 48, shown in FIG. 5, configured to be filled with an active agent. A fill valve 50 is coupled to the shell 46 and allows the active agent to fill the reservoir 48 from a filling mechanism (not shown), in a manner similar to how the reservoir 24 was filled, as discussed above in relation to FIG. 2.

FIG. 5 illustrates a cross sectional view of the intragastric volume occupying device 44 shown in FIG. 4. The reservoir 48 is shown to be positioned within the shell 46. The output device 22, the controller 28, and the tube 20 may each be configured similarly as the respective devices shown and discussed in relation to FIGS. 1 and 2.

The intragastric volume occupying device utilized in the present invention may therefore include a variety of shapes and mechanisms capable of occupying volume within a patient's stomach. As shown in FIGS. 1 and 2, the intragastric volume occupying device has a substantially spherical shape. As shown in FIGS. 4 and 5, the intragastric volume occupying device has a flattened disk-like shape. In one embodiment, intragastric volume occupying devices may be used to take up space in the patient's stomach that include foams, magnets, devices that expand due to a chemical reaction, nitinol clips, expandable posts, expandable stents, or a buckyball construction. Any of the embodiments discussed throughout this application may be fixed to the stomach via sutures, staples, adhesives, fixation clips, tacks or ports.

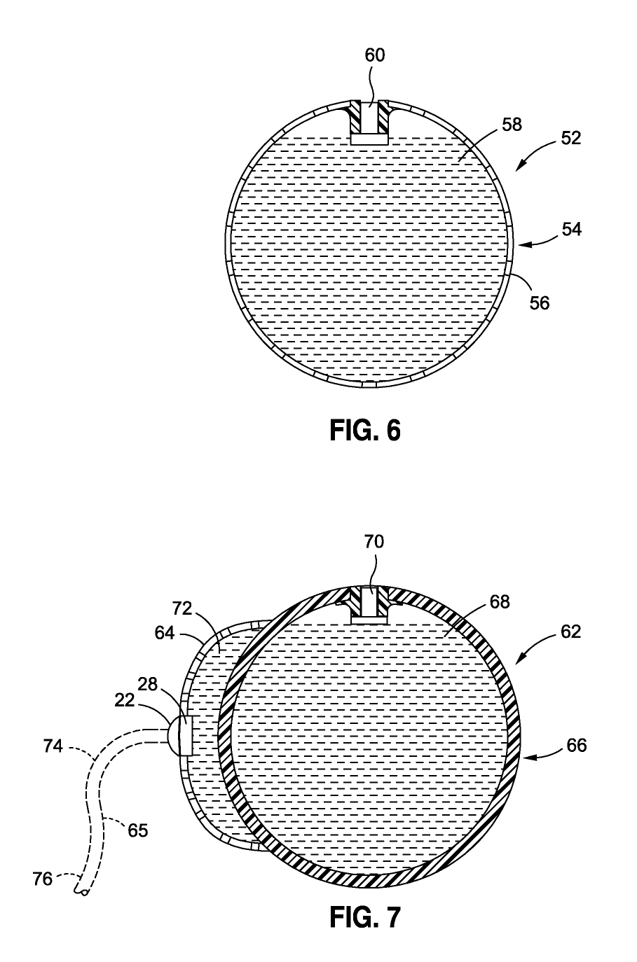

FIG. 6 illustrates an embodiment of an intragastric volume occupying device 52 including a semi-permeable membrane 56. The semi-permeable membrane 56 forms a shell 54 that defines the reservoir 58. A fill valve 60 is incorporated with the shell 54 to allow active agent to fill the reservoir 58. The semi-permeable membrane 56 may comprise a silicone or nanostructure material capable of selectively diffusing the active agent through the membrane 56. The membrane 56 serves as an outlet for the reservoir 58, allowing the active agent to diffuse through the membrane 56 based on a concentration difference, or mass action, of the active agent on one side of the membrane 56, in relation to the concentration on the other side of the membrane 56.

In the embodiment shown in FIG. 6, the semi-permeable membrane 56 may be configured to contact a portion of the patient's stomach and allow the active agent to diffuse into the local tissues and fluids within the patient's stomach. Because the semi-permeable membrane 56 serves as the outlet of the reservoir 58, an outlet device, for example, the outlet device 22 shown in FIGS. 1, 2, 3 and 5 is unnecessary.

FIG. 7 illustrates an embodiment of an intragastric volume occupying device 62 in which a semi-permeable membrane 64 forms only a portion of the shell 66 that surrounds a fill reservoir 68 and an active agent reservoir 72. In this embodiment, the semi-permeable membrane 64 surrounds the active agent reservoir 72. The semi-permeable membrane 64 allows the active agent to diffuse from the active agent reservoir 72 and contact a portion of the patient's body. Similar to the embodiment shown in FIG. 3, as the active agent exits the active agent reservoir 72, the size of the fill reservoir 68 will not decrease.

FIG. 7 additionally illustrates an embodiment in which a portion of a tube 74 extending from the active agent reservoir 72 includes a semi-permeable membrane 65. The active agent may diffuse from the tube 74 as the active agent passes through the tube 74, to be dispensed to a portion of the patient's body. In addition, in one embodiment, an end 76 of the tube 74 may be closed, such that the active agent cannot pass through the end 76 of the tube 74. Rather, the active agent may only exit the tube 74 through the semi-permeable membrane 65. In this embodiment, the tube 74 may be positioned as desired, for example, in the patient's upper intestines, and the active agent may slowly diffuse through the membrane 65, and into the local tissues of the upper intestines. The membrane 65 may be located in specific regions of the tube 74 to ensure that the active agent is diffused in a specific location of the body or intestines.

In addition, the outlet device 22 and the controller 28 shown in FIG. 7 may be configured similarly as the outlet device 22 and the controller 28 shown in FIGS. 2, 3 and 5.

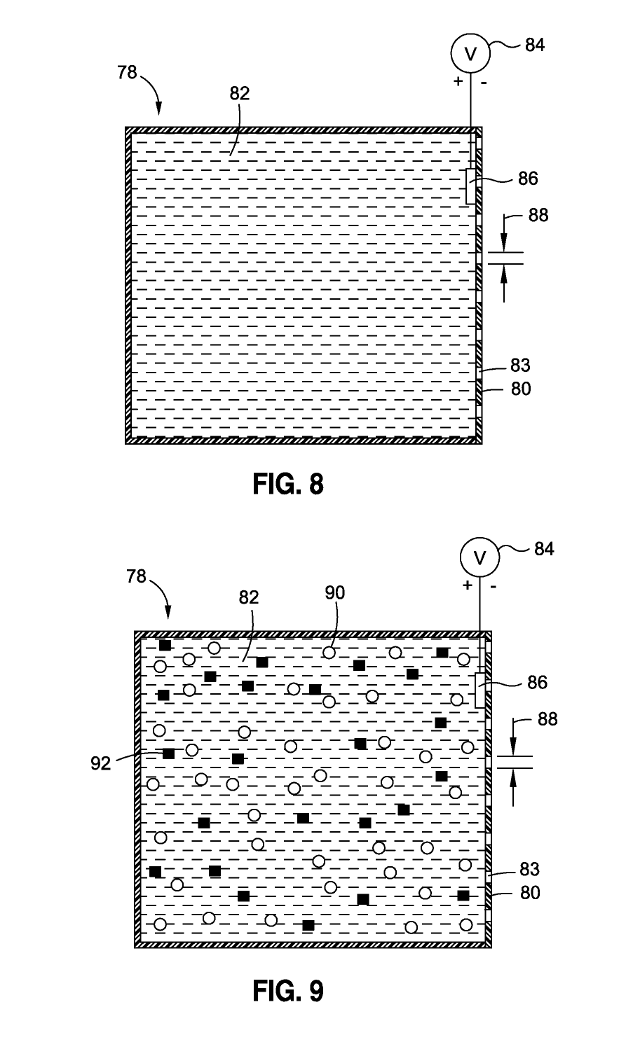

FIG. 8 illustrates a schematic representation of a reservoir 78 having a semi-permeable membrane 80. The reservoir 78 may represent any of the reservoirs discussed in relation to FIGS. 6-7, or any portion of an intragastric volume occupying device system having a semi-permeable membrane, discussed in relation to FIGS. 6-7. The reservoir 78 includes a central chamber or housing, configured to contain an active agent 82, represented by dashed lines in FIG. 8. An outer surface of the reservoir 78 comprises a semi-permeable membrane 80, having a series of pores 83 for the active agent 82 to pass through and exit the reservoir 78. The active agent 82 may diffuse through the semi-permeable membrane 80 based on a diffusive force, caused by a concentration differential of the active agent 82 on one side of the membrane 80 in relation to the other side of the membrane 80. The rate of diffusion may depend in part on the porosity of the membrane 80, the diffusibility of the active agent across the surface of the membrane 80, and the surface area to volume ratio of the reservoir 78. Such design features of the reservoir 78 may be varied to produce a desired effect.

In one embodiment, the passage of the active agent 82 from one side of the membrane 80 to the other may be aided or hindered by use of a voltage source 84 and an electrode 86, utilized in combination with the reservoir 78. The voltage source 84 may be powered inductively from a remote device and/or through a battery (not shown) that may be charged prior to implantation of the implantable system or charged inductively after implantation, through appropriate means. The electrode 86 may be positioned on either side of the membrane 80, or on both sides of the membrane 80, or generally within the reservoir 78 as desired. The voltage source 84 and the electrode 86 operate to form an electric charge on one side of the membrane 80, or on both sides of the membrane 80. The charge may enhance or impede the diffusion of the active agent from the reservoir 78 to the patient's body, as the active agent 82 may comprise molecules having a net charge, or a polarity. Based on the charge of the molecules forming the active agent, the electrode 86 may cause a charge to be formed on either side of the membrane 80 that either draws the active agent 82 out from the reservoir 78, or serves to keep the active agent 82 within the reservoir 78, through an electric force.

The desired polarity of the charge formed on either side of the membrane 80 will depend on the polarity of the molecules that were selected to comprise the active agent 82. In one embodiment, the net charge formed by the electrode 86 on either side of the membrane 80 may be varied as desired by the voltage source 84. In this embodiment, the voltage source 84 may be a variable voltage source. The voltage output by the voltage source 84, and the polarity of the net charge formed by the voltage source 84, may be controlled by, for example, the controller 28 discussed in relation to FIGS. 2, 3, 5 and 7. The controller 28 may be appropriately configured to instruct the voltage source 84 to produce a charge in response to a signal from the sensor 14 (shown in FIG. 1), or the external control device 30 (shown in FIG. 17). For example, the controller 28 may receive a signal from the sensor 14, directing the voltage source 84 to increase or decrease the strength, or polarity, of the net charge formed on either side of the membrane 80. If the sensor 14 detects a low hormone level in the patient, the sensor 14 may send a signal to the controller 28 instructing the controller 28 to increase the flow of the active agent 82 through the membrane 80. The controller 28 may instruct the voltage source 84 to increase the net charge, or vary the polarity of the charge, on one side of the membrane 80, if such an action will increase the flow of the active agent 82 through the membrane 80. A signal received from the external control device 30 may produce a similar result.

In one embodiment, the controller 28 may be configured to control the voltage source 84 to increase or decrease the strength or polarity of the net charge formed on either side of the membrane 80, according to a schedule. The controller 28 may be pre-programmed with a control schedule that defines the times the voltage source 84 will enhance or impede diffusion of the active agent 82 through the membrane 80. The control schedule may be configured to enhance or impede the diffusion of the active agent 82 through the membrane 80 at specified times during the day. For example, the control schedule may include a schedule of meal times. The amount of active agent 82 dispensed may increase at the defined meal times. In addition, the control schedule may be set to reduce the flow of the active agent 82 from the reservoir 78 during times when food consumption is not likely, for example, during times when the patient is likely to be sleeping. The control schedule may be configured to be varied or reprogrammed telemetrically after the controller 28 has been implanted. The control device 30, discussed in relation to FIG. 17, may be used to reprogram the controller 28.

In one embodiment, a pressure sensor may be incorporated with the reservoir 78. The pressure sensor may be capable of detecting a force exerted against the reservoir 78 by the patient's stomach or by food present in the patient's stomach. The pressure sensor may send a signal to the voltage source 84 to increase the flow of the active agent 82 through the membrane 80 in response to the force applied to the reservoir 78.

The response of the voltage source 84 to a signal from the sensor 14, or the external control device 30, or the pressure sensor, may be varied as desired (e.g., the voltage source 84 may decrease the net charge on one side of the membrane 80, if doing so would increase flow of the active agent 82 through the membrane 80). In one embodiment, the electrode 86 may equivalently comprise a plurality of electrodes, positioned throughout, and/or external to the reservoir 78, if equivalent operation results.

In one embodiment, the voltage source 84 and the electrode 86 may be utilized to vary the size 88 of the pores 83 of the membrane 80. The membrane 80 may be made of a material structured to contract or expand in response to an electric voltage applied to the membrane 80. The material of the membrane 80 is composed such that the pore size 88 depends on the voltage being applied to the membrane 80, for example, the material may comprise an electroactive polymer having a series of pores. The size 88 of the pores 83 depends on the presence of a voltage applied by the electrode 86, which may either increase or decrease the size 88 of the pores 83. The active agent 82 may either more easily, or less easily flow, through the membrane 80, if a voltage is applied to the membrane 80 by the electrode 86. In one embodiment, the voltage source 84 may be a variable voltage source 84, and may be controlled by the controller 28 discussed in relations to FIGS. 2, 3, 5 and 7. The controller 28 may operate in response to a signal produced by the sensor 14 (shown in FIG. 1) or an external control device 30 (shown in FIG. 17). For example, if the sensor 14 detects a low hormone level in the patient, the voltage source 84 may appropriately increase or decrease the voltage applied to the membrane 80, to increase the pore size 88, and increase the flow of the active agent 82 from the reservoir 78.

In one embodiment, the semi-permeable membrane 80 may be configured to vary the pore size 88 without the use of the voltage source 84 and the electrode 86. In this embodiment, the semi-permeable membrane 80 may be made from a material that varies in pore size 88 automatically in response to an environmental condition, based on the material properties of the membrane 80. The material may comprise polyethylene, or polyethylene filled with SiO₂. Such desirable materials are discussed in the publication, “Response of Filled Polyethylene Membranes to the Changes in the Environmental Conditions,” M. A. Islam and N. D. Nikolov, volume forty-five, issue 8, Journal of Applied Polymer Science, the entirety of which is incorporated by reference. The environmental condition may comprise a biological property, for example, a pH level, a temperature, or an internal pressure of the patient. The material of the membrane 80 may be selected such that the pore size 88 will automatically adjust in response to the environmental condition, and will adjust to a desired degree in response to the environmental condition. Thus, for example, if a pH level in a certain level is present within a response range of the membrane 80, the pore size 88 may increase or decrease to vary the flow of the active agent 82, as desired.

FIG. 9 illustrates an embodiment of the reservoir 78 shown in FIG. 8, which utilizes biological organisms 90 to produce the active agent 82. The biological organisms 90 are represented in FIG. 9 as circles, and may comprise any biological organism, preferably at the size of a microbe, or as would otherwise fit into a reservoir 78 for implantation within a patient's body. The biological organisms 90 may preferably comprise any cell or combination of zoograph cells, allograph cells, autograph cells, bacterium, algae, or yeast. The biological organisms 90 are stored within the reservoir 78 and are sustained by a nutrient media 92, represented in FIG. 9 as squares. The reservoir 78 is configured in a manner to properly store and keep the biological organisms 90 alive for an extended period of time. For example, the pores 83 are sized such that the biological organisms 90 cannot pass through the membrane 80, yet the active agent 82 produced by the biological organisms 90 may still pass through the pores 83. The pores 83 are preferably sized to be as small as possible while still allowing the diffusion of the agent across the membrane 80. Enough nutrient media 92 is supplied to the reservoir 78 to sustain the biological organisms 90 for an extended period of time. The internal volume of the reservoir 78 and nature of the nutrient media 92 selected should optimize the longevity, reproductive capabilities, health, and number of biological organisms 90. The reservoir 78 is designed to be safe from rupture. In one embodiment, the biological organisms 90 may be contained within microspheres to more effectively protect the biological organisms 90. The microspheres may be placed directly into the patient's body, without the use of the reservoir 78. In one embodiment, the microspheres may be injected intravenously into the patient's body.

The biological organisms 90 may be preferably sustained within the reservoir 78 for a period of no less than six months. A lifetime of up to thirty years may be reached. The sustainable duration of the biological organisms 90 may be varied, based on the scope of treatment desired by the patient, or the severity of the patient's obesity problem. The reservoir 78 may otherwise be configured in any manner discussed in reference to FIG. 8, including an embodiment in which a net charge is produced on a side of the membrane 80 to enhance or impede flow of the active agent, or an embodiment in which the size of the pores 83 is variable due to an applied voltage or environmental factors. The biological organisms 90, the active agent 82, and the nutrient media 92 may be replenished by any method discussed throughout this disclosure, including replenishment through an access port.

The biological organisms 90 utilized in the reservoir 78 may comprise bacteria, due to the relatively non-complex structure of bacterial DNA. The bacteria may be engineered to produce a desired active agent 82 as a result of biological engineering of the bacteria's DNA. Prior to the bacteria being implanted in the reservoir 78, a plasmid vector may be introduced into the bacteria containing a strand of DNA that will cause the bacteria to produce the desired active agent 82. The plasmid vector may be formed by splicing a desired sequence of DNA into the plasmid vector. The DNA sequence may be capable of producing the agent of interest, for example, the CCK-8 (cholecystokinin octapeptide) sequence may be used if desired. The DNA sequence is spliced into a vector having the appropriate promoter section. The vector may comprise a bacteriophage vector, a raboviral vector, a lentiviral vector, a plasmid vector, a herpes simplex viral vector, a semliki forest viral vector, a vesicular stomatitis viral vector, a baculoviral vector, an autographa californica nuclear polyhedrosis viral vector, and a ribonucleic acid interference (RNAi) via small interfering ribonucleic acids (siRNA). Other vectors may be utilized as desired, to produce an equivalent result. Once the vector has been introduced to the bacteria, the bacteria act as a self-replicating carrier of a vector which codes for the desired active agent. After the vector has been incorporated into the bacterial DNA, the bacteria containing the modified, or chimeric, strand of DNA will be replicated, amplified and reproduced. Methods of producing recombinant DNA are discussed in “AN INTRODUCTION TO GENETIC ANALYSIS” by Anthony Griffiths, Jeffery Miller, David Suzuki, Richard Lewontin, and William Gelbert. Further information may also be found in “MOLECULAR CELL BIOLOGY” by Harvey Lodish, Arnold Berk, Paul Matsudaira, Chris Kaiser, Monty Krieger, Matthew Scott, S. Lawrence Zipursky, and James Darnell. In one embodiment, electrical energy may be applied to the bacteria to weaken the cell wall of the bacteria. A plasmid is then introduced into the bacterial cell, having a DNA sequence that codes for a desired active agent. The bacterium will express the genes in the plasmid and produce the desired active agent.

Once a stable colony of chimeric bacteria are produced, the bacteria will then be introduced into the reservoir 78, either prior to implantation of the reservoir 78 within the patient's body, or after the reservoir 78 has been implanted. The bacteria may be introduced into the reservoir 78 through a fill valve, for example, one of the fill valves discussed in various embodiments disclosed throughout this disclosure. The active agent produced by the bacteria is selected such that no significant adverse symptoms are produced for the patient. The active agent is introduced to the patient at a rate sufficient to produce the desired treatment effect. In an embodiment wherein cholecystokinin octapeptide is utilized as the active agent, the desired rate of diffusion into the patient's body may be 1.6 picomole per liter per minute. Such a diffusion rate may produce a desired concentration of cholecystokinin octapeptide within the patient's body of 4 +/−0.5 picomoles per liter when the patient is fasted, and 8 +/−1.5 picomoles per liter when the patient is fed. In an embodiment, wherein peptide-tyrosine-tyrosine (3-36) (PYY 3-36) is utilized as the active agent, the desired rate of diffusion into the patient's body may be 0.3182 picomole per liter per minute. Such a diffusion rate may produce a desired concentration of peptide-tyrosine-tyrosine (3-36) within the patient's body of 11 +/−1 picomoles per liter when the patient is fasted or fasting and 20 +/−1 picomoles per liter when the patient is fed. Such diffusion rates and body concentrations are approximate, and may be varied as desired.

A benefit of utilizing the biological organisms 90 to produce the active agent 82 is that the active agent 82 may be replenished within the patient's body over an extended duration of time. A quantity of new active agent 82 is produced by the biological organisms 90 during the treatment period of the patient, rather than a single quantity of the active agent 82 being inserted into the reservoir 78 by a physician and then remaining in the patient's body, and being exposed to the internal body heat of the patient, for an extended duration of time. The biological organisms 90 may extend the effective life of the active agent 82 dispensed into the patient's body. In addition, the biological organisms 90 may reduce the number of times the reservoir 78 must be refilled.

The embodiments shown in FIGS. 6-9 that utilize a semi-permeable membrane may be incorporated with any of the embodiments shown throughout this disclosure. The semi-permeable membrane may allow for a slow, for example, constant, diffusion of the agent into the body from other locations in the body.

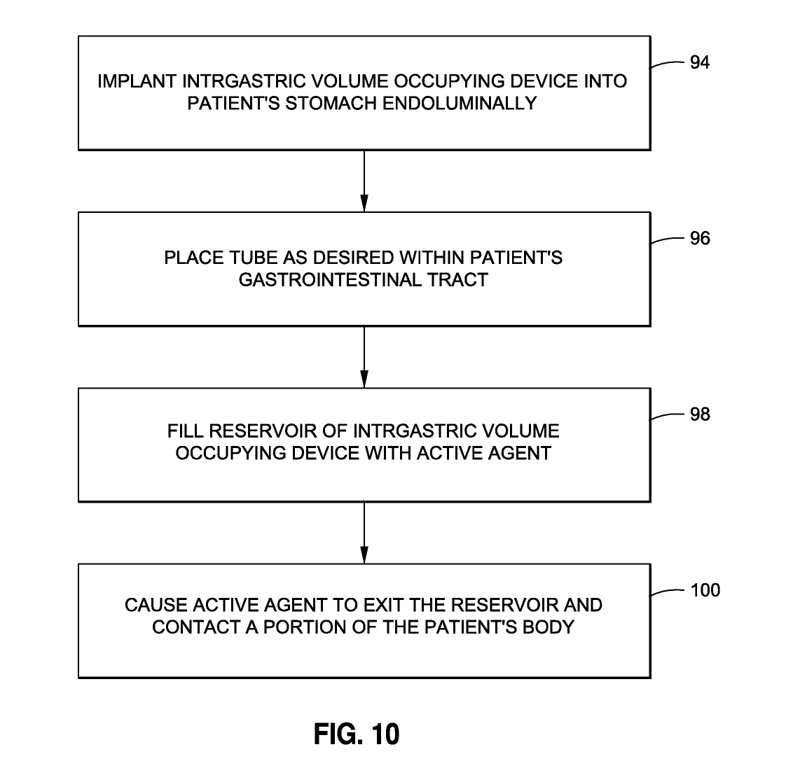

FIG. 10 illustrates an exemplary method for the treatment of obesity utilizing any of the intragastric volume occupying device system embodiments shown in FIGS. 1-9. In step 94, an intragastric volume occupying device is implanted within the patient's stomach endoluminally, or through endoluminal means. Endoluminal tools are utilized to place the intragastric volume occupying device into the patient's stomach. The intragastric volume occupying device may be inserted into the patient's stomach in a deflated state.

In step 96, a tube, for example, a tube 20, 74, shown in FIG. 1 or 7, may be placed as desired within the patient's gastrointestinal tract. The tube may be placed within the patient's stomach using endoluminal tools. In one embodiment, an end of the tube may be positioned within the patient's upper intestines.

In step 98, a reservoir, for example a reservoir 24, 40, 48, 58 or 72, shown in FIG. 2, 3, 5, 6 or 7, may be filled with an active agent. The reservoir may be filled through a fill valve coupled to the intragastric volume occupying device, or through other appropriate means. The reservoir may be filled with an active agent in fluid form, or an active agent and saline, or a combination of an active agent and any other fluid, as desired. In an embodiment in which the intragastric volume occupying device includes both an active agent reservoir and a fill reservoir, for example, as shown in FIG. 3, the fill reservoir may also be filled with an appropriate fluid, such as saline. A filling mechanism may be utilized to fill either the reservoir with fluid, including a fluid containing an active agent. For the embodiment shown in FIG. 9, the filling step may further comprise placing biological organisms and/or nutrient media into the reservoir. In one embodiment, the reservoir may be pre-filled with an amount of active agent prior to implantation. In an embodiment in which the intragastric volume occupying device comprises a balloon, the balloon may be filled to a range of between about 400 cc to 700 cc of fluid, with a typical fill volume of about 500 cc. In embodiments of the present invention, the fill volume may be varied as desired.

In step 100, the physician or patient may cause the active agent to be dispensed from the reservoir, through any of the means discussed throughout this disclosure. For example, the physician may activate a pump to dispense the active agent from the reservoir. In addition, the physician may implant and activate a biological sensor, capable of acting in a feedback loop with the reservoir, as discussed in relation to FIGS. 1 and 2. The active agent is dispensed to contact the desired portion of the patient's body.

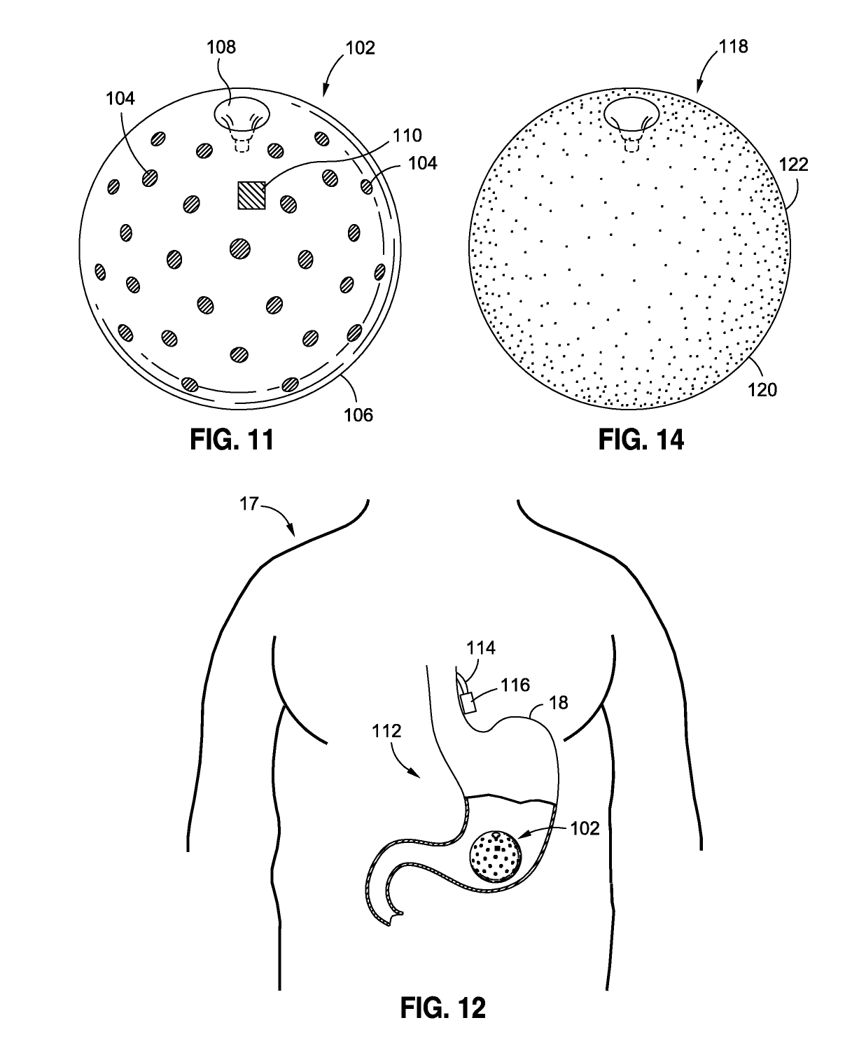

FIG. 11 illustrates an embodiment of an intragastric volume occupying device 102 having a plurality of electrodes 104 configured to apply electrical energy, or stimulation, to the patient's stomach. The intragastric volume occupying device 102 may include a shell 106 having a substantially spherical shape, and a fill valve 108, which allows the device 102 to be filled with an appropriate filling fluid. The electrodes 104 are positioned on the surface of the shell 106, and are capable of contacting the interior of the patient's stomach, to apply electrical stimulation directly to the patient's stomach. The intragastric volume occupying device 102 may further include an electrical control device 110 capable of containing instructions in a memory, which are executed by a processor. The electrical control device 110 may also include a transmitter and a receiver, the receiver being capable of receiving instructions from an external transmitter sent wirelessly, to allow the electrical control device 110 to be programmed after implantation. The electrical control device 110 may also be programmable prior to implantation. The instructions stored in the memory may also cause the electrodes 104 to apply electrical stimulation based on a timer or a schedule stored in the memory.

The electrical control device 110 may additionally include a pressure sensor, capable of sensing gastric activity of the patient. For example, the sensor may detect forces exerted against the sensor by the patient's stomach, or by food contained within the patient's stomach. The electrical control device 110 may be positioned on the surface of the volume occupying device 102 to detect a force exerted against the volume occupying device 102 in response to gastric activity. The electrical control device 110 may then instruct the electrodes 104 to apply electrical energy to the patient's stomach, in response to the force detected by the control device 110. The electrical control device 110 may be powered, either inductively from a remote device or through a battery (not shown) that may be charged prior to implantation of the intragastric volume occupying device 102 or charged inductively after implantation, through appropriate means.

The application of electrical energy to the patient's stomach, used in conjunction with an intragastric volume occupying device 102, may serve to promote satiety signals delivered to the patient's brain. The electrical energy may stimulate local nerves that are normally only stimulated when food is present in the patient's stomach. The electrical impulses may strengthen the intensity of these signals to indicate to the patient's brain that more extensive food consumption has occurred than has actually occurred. The patient may then feel full more quickly, and will reduce food consumption sooner. The use of a pressure sensor in the electrical control device 110 may assure that the enhanced satiety signals are produced when most relevant for the patient, namely, during times of food consumption.

FIG. 12 illustrates an embodiment of an intragastric volume occupying device system 112 that includes the intragastric volume occupying device 102 shown in FIG. 11, and a plurality of electrodes 114 that couple to the lower third of the patient's esophagus. The electrodes 114 are fixed to the patient's esophagus by known means, including an adhesive, barbs, sutures, or similar other means. The electrodes 114 extend from an electrical control device 116 being separate from the electrical control device 110 discussed in relation to FIG. 11. The electrodes 114 may comprise thin, wire-like projections extending from the electrical control device 116 in a direction away from the patient's stomach. The electrical control device 116 may be configured similarly as the electrical control device 110 on the intragastric volume occupying device 102. The electrical control device 116 may be capable of powering the electrodes 114 and causing the electrodes 114 to apply electrical stimulation to the lower third of the patient's esophagus.

The electrical control device 116 may be controlled wirelessly from the control device 110 on the intragastric volume occupying device 102. For example, the electrical control device 116 may be configured to receive a signal from the intragastric volume occupying device 102, causing the electrodes 114 to apply electrical energy to the lower third of the patient's esophagus. The electrodes 114 may be configured to apply electrical energy to the lower third of the patient's esophagus in response to a force detected by the electrical control device 110. The force may have been exerted against the patient's stomach by the patient's stomach or by food in the patient's stomach. In one embodiment, the electrical control device 116 may be controlled wirelessly from an external controller, for example, the external control device 30 shown in FIG. 17.

A benefit of placing the electrode 114 along the lower third of the esophagus, or lower thoracic esophagus, is to enhance the effect of the electrical stimulation applied to the patient's body. Recent studies suggest direct stimulation to the lower third of the esophagus may produce enhanced stimulation of local nerves, including the vagus nerve, which will enhance the production of satiety signals. The stimulation of the lower third of the esophagus, as opposed to direct simulation of the vagus nerve along other portions of the patient's body, for example, the patient's stomach, offers an improvement over prior known electric stimulation methods. In addition, the use of electrodes along the lower third of the esophagus, in combination with an intragastric volume occupying device positioned in the patient's stomach, produces a superior combination of obesity treatments over electrical stimulation alone, or intragastric volume occupying device treatment alone, or electric stimulation of other portions of the vagus nerve, including along the stomach. In one embodiment, the electrode 114 may be placed along the vagus nerve in a position not along the lower third of the esophagus. However, it is understood the position of the electrode 114 along these other portions of the vagus nerve may not include the therapeutic effects of the electrodes 114 placed along the lower third of the esophagus.

In one exemplary method of operation, the electrode 114 is inserted laparoscopically within the patient's body. The electrode 114 is then coupled to the lower third of the patient's esophagus, to offer superior production of satiety signals in response to electrical stimulation. The electrode 114 may be powered by an electrical control device 116. The electrical control device 116 may be wirelessly controlled by an electrical control device 110 integrated with the intragastric volume occupying device.

Any of the embodiments shown in FIGS. 11-12 may be incorporated with any other embodiment shown throughout this application. For example, electric stimulation in combination with the use of a reservoir of the active agent may serve to greatly enhance the production of satiety signals produced in an individual's body. Any combination of treatments may be used as desired to enhance the treatment of obesity.

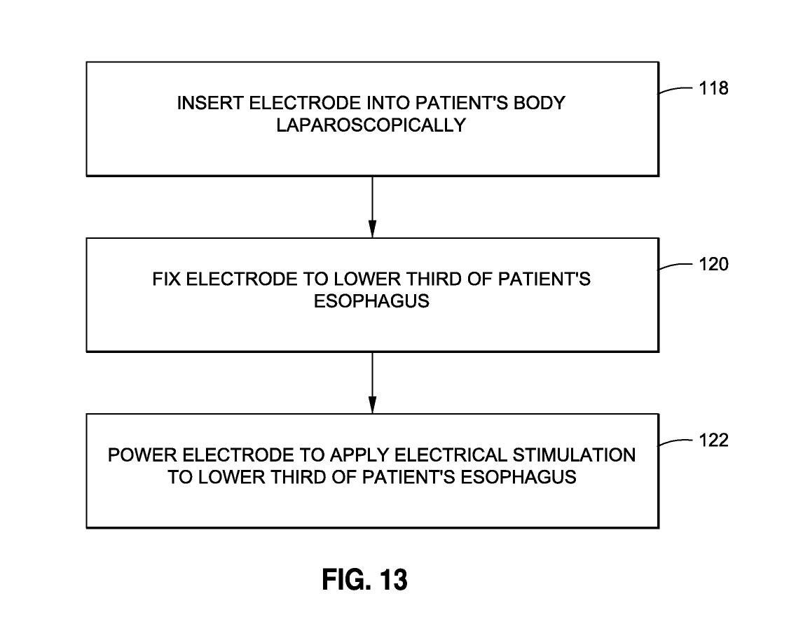

FIG. 13 illustrates an exemplary method for the treatment of obesity utilizing the electrical stimulation device embodiment shown in FIG. 12. In step 118, an electrode is inserted within a patient's body laparoscopically, or through laparoscopic means. Laparoscopic tools are utilized to insert the electrode into the patient's body. The electrode may be utilized in combination with an intragastric volume occupying device, for example, as shown in FIG. 12. A combination of intragastric volume occupying device treatment and electric treatment to the lower third of the patient's esophagus offers superior therapeutic properties than an intragastric volume occupying device used alone, or in combination with electric treatment to other portions of the patient's body.

In step 120, the electrode is fixed to the lower third of the patient's esophagus. The electrode may be fixed to the patient's esophagus using barbs, tacks, sutures, adhesives, or the like. If the electrode is coupled to an electrical control device, for example, the electrical control device 116 shown in FIG. 12, then the electrical control device may be implanted prior to, at, or after, this step. The electrode may comprise a wire-like projection extending from the electrical control device and connecting to the esophagus. The electrode may be positioned along a portion of the patient's vagus nerve extending along the lower third of the esophagus. In an embodiment including a plurality of electrodes, each electrode may be positioned in sequence along the vagus nerve, or other portions of the lower third of the esophagus. In an embodiment including an intragastric volume occupying device, for example, the intragastric volume occupying device 102 shown in FIGS. 11 and 12, the intragastric volume occupying device may be inserted into the patient's body prior to, at, or after, this step. The intragastric volume occupying device may include an electrical control device, for example, the electrical control device 110, which is capable of wireless communication with the electrical control device coupled to the electrode.

In step 122, the electrode is powered to apply electrical stimulation to the lower third of the patient's esophagus. Such power may be delivered via a battery charge, or an inductive charge. The electrical control device, for example, the electrical control device 116, may be powered at this step to allow the electrical control device to cause the electrode to deliver electric stimulation to the lower third of the esophagus.

FIG. 14 illustrates an embodiment of the present invention including an intraluminal volume occupying device 118 having an ancillary device 120 incorporated into the structure 122 of the intraluminal volume occupying device 118. The ancillary device 120 may include a coating of an active agent placed on the structure 122 of the intraluminal volume occupying device 118.

In some embodiments, the ancillary device 120 comprises a composition incorporated into the intraluminal volume occupying device 118. The composition may comprise a matrix material and an active agent combined with the matrix material, providing a coating of the active agent over the surface of the intraluminal volume occupying device 118. The matrix material may be a biodegradable material, referred to equivalently as a bioerodible material, for example, a bioerodible polymer which, during erosion thereof in the body, releases the active agent from the composition, for example, in a controlled manner, for example, in a time-released fashion.

Alternatively, the ancillary device 120 may comprise a non-bioerodible material structured to facilitate release of an active agent into the body. In some embodiments, the ancillary device 120 includes structures for containing and releasing the active agents, for example, in a controlled manner. In one embodiment, combinations of bioerodible and non-bioerodible materials for containing and releasing active agents are also contemplated.

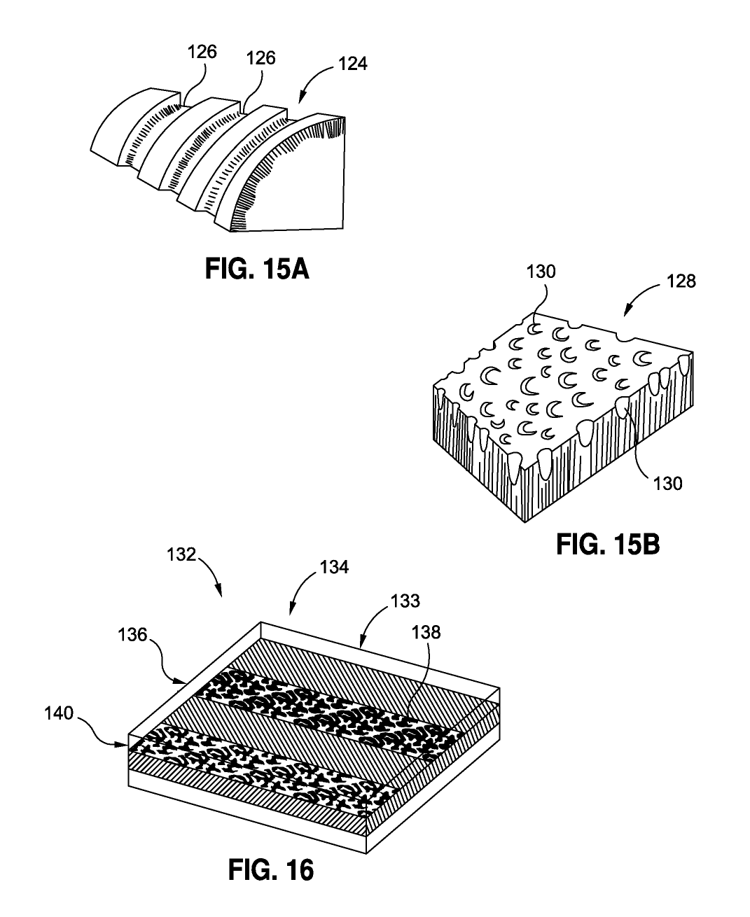

FIG. 15A illustrates an embodiment of an ancillary device 124 including recessions, pores or grooves capable of containing an agent. The ancillary device 124 may include one or more of the features of the ancillary device 120 described herein.

The ancillary device 124 comprises a polymer surface having one or more indentations or grooves 126 capable of containing or holding a satiety inducing agent, or a composition containing a satiety inducing agent, for example, in solid, gel, powder, paste or other form. The ancillary device 124 may form a coating of active agent over the surface of an intragastric volume occupying device.

Turning now to FIG. 15B, alternatively or additionally, an ancillary device 128 comprises a polymer surface having a porous or other irregular structure, wherein pores 130 are capable of containing or holding an active agent, or a composition such as a matrix material containing an active agent. The ancillary device 124 may form a coating of the active agent over the surface of an intragastric volume occupying device.

The ancillary devices 120, 124, 128 may be made of any suitable, biocompatible material, for example, any suitable material approved by the Food and Drug Administration (FDA) for use in humans, for example, as approved for long term administration of agents and long term placement or implantation in the body. In one embodiment, the material is ethylene vinyl acetate (EVA).

Referring now to FIG. 16, in some embodiments of the invention, the ancillary device 132 comprises a film or membrane 134 which makes up a surface coating on an intragastric volume occupying device, for example, a surface of the intragastric volume occupying device which contacts the stomach when the volume occupying device is appropriately positioned. The film 134 is capable of releasing a satiety inducing agent from the intragastric volume occupying device and into the patient, for example, at a controlled rate.

For example, the film 134 may comprise a first membrane layer 133 and a second membrane layer 136. The film 134 may further comprise a composition containing a satiety inducing agent, wherein the composition is located adjacent, for example, between the first and second membrane layers 133, 136. The first and second membrane layers 133, 136 may comprise EVA or other suitable polymer or copolymer.

In the embodiment shown in FIG. 16, the film 134 further comprises first and second agent layers 138, 140 which are made up of a composition containing a satiety inducing agent. The first and second agent layers 138, 140 are disposed in an alternating fashion with respect to the first and second membrane layers 133, 136. The membrane layers 134, 136 may have a known diffusion rate relative to the selected satiety inducing agent.

The film 134 is effective to control dosage and delivery of the agents to the patient. The film 134 may therefore have a desired porosity and/or be made of a suitable material so as to provide a controlled release of the agent.

For example, each of the ancillary devices described herein, for example, devices 120, 124, 128 and 132 may be structured to provide effective concentrations of the agent for about six months, or for about one year, about two years, or about three years or more. In some embodiments, the devices 120, 124, 128 and 132 are structured to provide a sustained release rate, for example, of three years followed by a gradually decreasing release rate over the next about two to about three years. The duration of the effective concentration of the agent, and the release rate, may be varied as desired. Numerous release protocols are contemplated by the inventors, and are understood to fall within the scope of the present invention.

In one embodiment, the agent could also be applied to the intragastric volume occupying device via a slow release drug eluting coating similar to coatings used on cardiovascular stents such as the Cordis Sirolimus Drug eluting stent or the contraceptive device Norplant. The coating could be applied directly to the intragastric volume occupying device for a slow release of the drug into the body.

FIG. 17 illustrates an embodiment of an intragastric volume occupying device system 142 used for the treatment of obesity. The system 142 includes the sensor 14 and an external control device 30, which may be operated by the patient or by a physician, each equivalently referred to as the user in this disclosure. The system 142 preferably includes an intragastric volume occupying device 144, which may be configured similarly as the intragastric volume occupying device 12 shown in FIGS. 1 and 2. For example, the intragastric volume occupying device may include a reservoir configured to dispense an active agent. The use of an intragastric volume occupying device 144 in combination with the therapeutic actions discussed in relation to the system 142 (e.g., application of a patch, drinking of a liquid, etc.) is designed to treat obesity in the patient to a greater degree than a treatment solely involving an intragastric volume occupying device.

The external control device 30 may comprise a handheld device that may be carried by the patient, or may be used by the physician. The external control device 30 may also comprise any other electrical device used external to the patient, and capable of receiving and/or transmitting information to the sensor 14 (shown in FIG. 1). The external control device 30 may include a transmitter, a receiver, a processor and a memory. The external control device 30 may additionally include an alerting system, which may comprise an auditory alarm or notification, or a visual stimulation or notification, such as a light or a reading on a display screen, or a physical alerting system, including movement of the external control device 30, such as a vibration. The memory may store instructions, executed by the processor. The instructions may cause the control device 30 to perform any of the operations discussed throughout this disclosure. The external control device 30 may also include input means, such as a keypad, for the user to input instructions into the control device 30.

The receiver of the control device 30 may include an antenna capable of receiving signals sent from the sensor 14. The signal may provide information to a user, informing the user about the readings of the sensor 14. For example, the control device 30 may alert the user to take action in response to the signal sent from the sensor 14. The signal transmitted from the sensor 14 may indicate to the external control device 30 that a biological characteristic, such as a hormone level, is below a threshold value for the patient. The external control device 30 may then provide a notification, or publish certain responses to the user, for the user to take action, in response to the biological characteristic sensed by the sensor. The action preferably is effective to vary the biological characteristic sensed by the sensor. The notification, or publication, may utilize the alerting system, which may involve the sounding of an alarm, or a message presented on a display for the user to take action.

In response to the alert from the control device 30, the user may take a series of actions. Generally, the actions are designed to respond to the alert provided by the control device 30. For example, if the alert indicates a low hormone level in the patient, then the user may perform such actions that will increase the hormone level.

One such action may include injecting the patient with an active agent, possibly with a syringe. The injection may be a manual injection directly into the body of the patient. The control device 30 may alert the patient to inject the patient's body with a syringe during routine times. The injections may be performed under physician or patient control. The injections could occur routinely, or as advised by the control device 30.

Another action may include increasing the hormone level of the patient through a patch placed on the skin of the patient. The patch may have an active agent on one side of the patch, and may be capable of slowly diffusing the active agent through the patient's skin. The patch could be replaced routinely, or as advised by the control device 30.

Another action may include the patient inhaling an active agent, either through the nose or mouth or spraying an active agent into the nose or mouth. A nasal spray may allow the vaporous active agent to be applied to the nasal canal for immediate absorption. The active agent would be received by the patient closer to the satiety centers of the brain. An inhalant would allow the vaporous active agent to be absorbed into the lungs. The inhalant or spray could be administered routinely, or as advised by the control device 30.

Another action may include the patient drinking a liquid including an active agent. The active agent may be absorbed in the mouth, esophagus, or further down the gastrointestinal tract. A liquid may also be sprayed into the patient's mouth. The liquid could be administered routinely, or as advised by the control device 30.

Another action may include the patient swallowing a pill containing a desired active agent. In one embodiment, the pill may be coated to allow for slow, continuous or timed release. In one embodiment, the pill may be coated to react when in combination with a certain pH to allow it to pass into a specific location of the gastrointestinal tract. In one embodiment, the pill may have multiple mini-spheres of active agent coated with a variety of coatings controlled by pH to allow for the active agent to be released throughout the gastrointestinal tract. The pill could be administered routinely, or as advised by the control device 30.

Another action may include the user placing an orally received substance within the patient's mouth, including a film or gum that may introduce an active agent into the patient's body. The substance may be placed under the patient's tongue, and introduced into the patient's body through the local mucous membranes beneath the tongue. A chewing gum may allow the active agent to be absorbed by the mucous membranes within the patient's mouth. The orally received substance could be administered routinely, or as advised by the control device 30.

The actions, performed in combination with an intragastric volume occupying device therapy, will provide for a superior treatment of obesity during the duration of the treatment, in contrast to an intragastric volume occupying device treatment alone, or the actions performed alone.

The external control device 30 may be configured to select an appropriate alert in response to the signal sent from the sensor 14. For example, the control device 30 may be programmed to determine if the sensor 14 has indicated a hormone level is too high, and will alert the user to reduce hormone intake. In addition, the control device 30 may be programmed to determine if the sensor 14 has indicated a hormone level is too low, and will alert the user to increase hormone intake, in any form. The control device 30 may be configured to program the threshold detection level into the sensor 14, as discussed in relation to FIGS. 1 and 2.

In one embodiment, the control device 30 may be configured to instruct the user on which particular action to take, based on the readings of the sensor 14. For example, the control device 30 may select whether the patient should ingest a pill having a hormone, or chew a gum having a hormone, based on the signal sent from the control device 30. The particular action selected may be based on the degree to which the biological characteristic deviates from a threshold value, or based on a schedule of therapy designed for the patient, by, for example, the physician.

In one embodiment, the sensor 14 may be incorporated with an intragastric volume occupying device, to provide local readings of a desired biological characteristic for the patient. The sensor 14 may telemetrically send signals to the control device 30 using a transmitter integrated with the sensor 14.

The external control device 30 may be used with various other embodiments of systems for the treatment of obesity discussed throughout this disclosure. For example, the transmitter of the external control device 30 may be used to transmit signals to the controller 28, discussed in relation to FIGS. 2, 3, 5 and 7. The external control device 30 may be capable of causing the output device 22 to emit, or not emit, the active agent into the patient's body, in response to instructions sent from the external control device 30. The external control device 30 may be capable of setting the rate at which the output device 22 dispenses the active agent from the reservoir. The external control device 30 may be capable of programming an active agent dispersion schedule into the controller 28. In addition, the sensor 14, the external control device 30, and the controller 28, may act in a closed loop, wherein the sensor 14 senses a biological parameter of the patient, and sends a signal to the external control device 30 that indicates to the user to take a specified action. The user may then instruct the controller 28 to distribute the active agent from the reservoir 78. In addition, the sensor 14 may cause the controller 28 to distribute the active agent from the reservoir without the intervention of the user. The external control device 30, however, may receive notification that the controller 28 is automatically distributing the active agent in response to the sensed biological parameter. The external control device 30 may give the user the opportunity to intervene, and prevent the automatic distribution of the active agent, or enhance the distribution of the active agent.

In one embodiment, the system 142 includes an external distribution device 146 that may be positioned external to the patient's body. The distribution device 146 may comprise a reservoir for holding an active agent that is capable of being injected into the patient's body, or pumped into the patient's body through a tube. The distribution device 146 may be capable of accurately metering the volume and rate at which the active agent is injected into the body. The external distribution device 146 may receive signals directly from the sensor 14 instructing the device 146 to distribute the active agent to the patient's body. The external distribution device 146 may automatically distribute the active agent to the body in response to this signal, or may produce an alert to the user instructing the user to take action. The user may cause the external distribution device 146 to dispense the active agent to the patient's body in response to the signal from the sensor 14. A benefit of the external distribution device 146 is that the active agent may be refilled by the patient, or physician, without having to insert endoluminal tools to inflate the intragastric volume occupying device. The patient could load and self-administrate the external distribution device 146.

In one embodiment, the actions taken in response to the alert from the control device 30 may be performed solely, or without prompting from the control device 30. For example, the patient may undergo an obesity treatment including the placement of an intragastric volume occupying device in the patient's stomach, in combination with the use of an active agent, that is introduced into the patient's body through a syringe, or a patch, or an inhalant or spray, or a liquid to be consumed by the patient, or a pill, or a film or a chewing gum introduced into the patient's mouth. The combination of the intragastric volume occupying device and the actions that introduce the active agent into the patient's body may produce a superior treatment for obesity, in comparison to an intragastric volume occupying device treatment alone. In one embodiment, a patient may treat obesity by performing the above-listed actions, including the use of an active agent that is introduced into the patient's body through a syringe, or a patch, or an inhalant or a spray, or a liquid to be consumed by the patient, or a pill, or a film or a chewing gum introduced into the patient's mouth. The actions may be performed without the use of an intragastric volume occupying device.

Any of the embodiments discussed in relation to FIG. 17 may be incorporated with any other embodiment shown throughout this disclosure. For example, a treatment involving insertion of chewing gum into the patient's mouth may be used in combination with electric stimulation, and/or a reservoir of the active agent positioned within the patient's body, and/or an ancillary device incorporated in any structure of the system. Any combination of treatments discussed throughout this disclosure may be used as desired to enhance the treatment of obesity.

### Example GLP-1

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes a procedure to have a volume occupying device implanted into his stomach, having a porous surface, or a slowly drug eluting membrane, or a dissolvable film, or small grooves, containing glucagon like peptide 1 (GLP-1) that is released at a rate to achieve plasma concentrations of [10-30 pMol/L]ₚ GLP-1 over a period of about 3-24 months. A reservoir containing glucagon like peptide 1 (GLP-1), according to any of the embodiments of this application may also be utilized. An injection, liquid, pill, spray, inhalant, patch, or oral substance such as a gum containing glucagon like peptide 1 (GLP-1) may also be used. The patient reports a marked suppression of appetite, and within 12 months, the patient has lost 58 pounds.

### Example of OXM

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes a procedure to have a volume occupying device implanted into his stomach, having a porous surface, or a slowly drug eluting membrane, or a dissolvable film, or small grooves, containing oxyntomodulin (OXM) that is released at a rate to achieve plasma concentrations of [105-150 pMol/L]ₚ OXM over a period of 3-24 months. A reservoir containing oxyntomodulin (OXM), according to any of the embodiments of this disclosure may also be utilized. An injection, liquid, pill, spray, inhalant, patch, or oral substance such as a gum containing oxyntomodulin (OXM) may also be used. The patient reports a marked suppression of appetite, and within 12 months, the patient has lost 58 pounds.

### Example of PYY

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes a procedure to have a volume occupying device implanted into his stomach, having a porous surface, or a slowly drug eluting membrane, or a dissolvable film, or small grooves, containing Peptide Y-Y (PYY) that is released at a rate to achieve plasma concentrations of [10-55 pMol/L]ₚ PYY over a period of 3-24 months. A reservoir containing Peptide Y-Y (PYY), according to any of the embodiments of this disclosure may also be utilized. An injection, liquid, pill, spray, inhalant, patch, or oral substance such as a gum containing Peptide Y-Y (PYY) may also be used. The patient reports a marked suppression of appetite, and within 12 months, the patient has lost 58 pounds.

### Example of PP

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes a procedure to have a volume occupying device implanted into his stomach, having a porous surface, or a slowly drug eluting membrane, or a dissolvable film, or small grooves, containing Pancreatic Peptide (PP) that is released at a rate to achieve plasma concentrations of [150-300 pMol/L]ₚ PP over a period of 3-24 months. A reservoir containing Pancreatic Peptide (PP), according to any of the embodiments of this application may also be utilized. An injection, liquid, pill, spray, inhalant, patch, or oral substance such as a gum containing Pancreatic Peptide (PP) may also be used. The patient reports a marked suppression of appetite, and within 12 months, the patient has lost 58 pounds.

### Example of Insulin

A 49 year old male patient, having a body weight of 322 pounds and a height of 5′11″, complains to his physician that he has tried unsuccessfully to lose weight over the past 15 years and is concerned about the effect his excess weight may have on his health. His physician suggests a procedure intended to provide the patient's body with better control over its blood sugars. At the physician's directive, the patient undergoes a procedure to have a volume occupying device implanted into his stomach, having a porous surface, or a slowly drug eluting membrane, or a dissolvable film, or small grooves, containing Insulin that is released at a rate to achieve plasma concentrations of [5-30 μU/mL]ₚ Insulin over a period of 3-24 months. A reservoir containing Insulin, according to any of the embodiments of this application may also be utilized. An injection, liquid, pill, spray, inhalant, patch, or oral substance such as a gum containing Insulin may also be used. The patient reports euglycemic effects, or better control by his body of blood sugars.

### Example of Leptin

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes a procedure to have a volume occupying device implanted into his stomach, having a porous surface, or a slowly drug eluting membrane, or a dissolvable film, or small grooves, containing Leptin that is released at a rate to achieve plasma concentrations of *[3-10 ng/mL]ₚ Leptin over a period of 3-24 months. A reservoir containing Leptin, according to any of the embodiments of this disclosure may also be utilized. An injection, liquid, pill, spray, inhalant, patch, or oral substance such as a gum containing Leptin may also be used. The patient reports a marked suppression of appetite, and within 12 months, the patient has lost 58 pounds. *In the case of a female patient the goal plasma concentrations would be [10-20 ng/mL]ₚ

### Example of Amylin

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes a procedure to have a volume occupying device implanted into his stomach, having a porous surface, or a slowly drug eluting membrane, or a dissolvable film, or small grooves, containing Amylin that is released at a rate to achieve plasma concentrations of [20-25 pMol/L]ₚ Amylin over a period of 3-24 months. A reservoir containing Amylin, according to any of the embodiments of this disclosure may also be utilized. An injection, liquid, pill, spray, inhalant, patch, or oral substance such as a gum containing Amylin may also be used. The patient reports a marked suppression of appetite, and within 12 months, the patient has lost 58 pounds.

### Example of CCK

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes a procedure to have a volume occupying device implanted into his stomach, having a porous surface, or a slowly drug eluting membrane, or a dissolvable film, or small grooves, containing Cholecystokinin (CCK) that is released at a rate to achieve plasma concentrations of [5-10 pMol/L]ₚ CCK over a period of 3-24 months. A reservoir containing Cholecystokinin (CCK), according to any of the embodiments of this disclosure may also be utilized. An injection, liquid, pill, spray, inhalant, patch, or oral substance such as a gum containing Cholecystokinin (CCK) may also be used. The patient reports a marked suppression of appetite, and within 12 months, the patient has lost 58 pounds.

### Example of CNTF

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes a procedure to have a volume occupying device implanted into his stomach, having a porous surface, or a slowly drug eluting membrane, or a dissolvable film, or small grooves, containing Ciliary neuro-trophic factor (CNTF) that is released at a rate to achieve plasma concentrations of [25-1300 pg/dL]ₚ CNTF over a period of 3-24 months. A reservoir containing Ciliary neurotrophic factor (CNTF), according to any of the embodiments of this application may also be utilized. An injection, liquid, pill, spray, inhalant, patch, or oral substance such as a gum containing Ciliary neuro-trophic factor (CNTF) may also be used. The patient reports a marked suppression of appetite, and within 12 months, the patient has lost 58 pounds.

### Example of CART

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes a procedure to have a volume occupying device implanted into his stomach, having a porous surface, or a slowly drug eluting membrane, or a dissolvable film, or small grooves, containing Cocaine-Amphetamine Regulated Transcript (CART) that is released at a rate to achieve plasma concentrations of [50-250 pM]ₚ CART over a period of 3-24 months. A reservoir containing Cocaine-Amphetamine Regulated Transcript (CART), according to any of the embodiments of this application may also be utilized. An injection, liquid, pill, spray, inhalant, patch, or oral substance such as a gum containing Cocaine-Amphetamine Regulated Transcript (CART) may also be used. The patient reports a marked suppression of appetite, and within 12 months, the patient has lost 58 pounds.

### Example of Ghrelin Inhibition/Antagonism

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes a procedure to have a volume occupying device implanted into his stomach, having a porous surface, or a slowly drug eluting membrane, or a dissolvable film, or small grooves, containing a drug that is released at a rate to achieve plasma concentrations of Ghrelin at [15-30 pg/mL]ₚover a period of 3-24 months. A reservoir containing Ghrelin blocker, according to any of the embodiments of this application may also be utilized. An injection, liquid, pill, spray, inhalant, patch, or oral substance such as a gum containing Ghrelin blocker may also be used. The patient reports a marked suppression of appetite, and within 12 months, the patient has lost 58 pounds.

### Example of NPY Inhibition/Antagonism

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes a procedure to have a volume occupying device implanted into his stomach, having a porous surface, or a slowly drug eluting membrane, or a dissolvable film, or small grooves, containing a drug that is released at a rate to achieve plasma concentrations of Neuro-peptide Y (NPY) at [65-95 pMol/L]ₚ over a period of 3-24 months. A reservoir containing Neuro-peptide Y (NPY) antagonists, according to any of the embodiments of this disclosure may also be utilized. An injection, liquid, pill, spray, inhalant, patch, or oral substance such as a gum containing Neuro-peptide Y (NPY) antagonists may also be used. The patient reports a marked suppression of appetite, and within 12 months, the patient has lost 58 pounds.

### Example of Orexin A Inhibition/Antagonism

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes a procedure to have a volume occupying device implanted into his stomach, having a porous surface, or a slowly drug eluting membrane, or a dissolvable film, or small grooves, containing a drug that is released at a rate to achieve plasma concentrations of Orexin A at [20-50 pg/mL]ₚ over a period of 3-24 months. A reservoir containing Orexin A antagonists, according to any of the embodiments of this disclosure may also be utilized. An injection, liquid, pill, spray, inhalant, patch, or oral substance such as a gum containing Orexin A antagonists may also be used. The patient reports a marked suppression of appetite, and within 12 months, the patient has lost 58 pounds.

### Example of AgRP Inhibition/Antagonism

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes a procedure to have a volume occupying device implanted into his stomach, having a porous surface, or a slowly drug eluting membrane, or a dissolvable film, or small grooves, containing a drug that is released at a rate to achieve plasma concentrations of AgRP at [1-16 ng/dL]ₚ over a period of 3-24 months. A reservoir containing AgRP antagonists, according to any of the embodiments of this disclosure may also be utilized. An injection, liquid, pill, spray, inhalant, patch, or oral substance such as a gum containing AgRP antagonists may also be used. The patient reports a marked suppression of appetite, and within 12 months, the patient has lost 58 pounds.

Although the invention has been described and illustrated with a certain degree of particularity, it is to be understood that the present disclosure has been made only by way of example, and that numerous changes in the combination and arrangement of parts can be resorted to by those skilled in the art without departing from the scope of the invention, as hereinafter claimed. For example, any of the obesity treatment methods, systems, and devices discussed throughout this disclosure may be used singularly, or in combination, as desired.

The foregoing disclosure is illustrative of the present invention and is not to be construed as limiting the invention. Although one or more embodiments of the invention have been described, persons skilled in the art will readily appreciate that numerous modifications could be made without departing from the spirit and scope of the present invention. It should be understood that all such modifications are intended to be included within the scope of the invention.

The terms “a,” “an,” “the,” and similar referents used in the context of describing the present invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the present invention otherwise claimed. No language in the specification should be construed as indicating any non-claimed element essential to the practice of the present invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, certain references have been made to patents and printed publications throughout this specification. Each of the above-cited references and printed publications are individually incorporated herein by reference in their entirety.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the present invention disclosed herein are illustrative of the principles of the present invention. Other modifications that may be employed are within the scope of the present invention. Thus, by way of example, but not of limitation, alternative configurations of the present invention may be utilized in accordance with the teachings herein. Accordingly, the present invention is not limited to that precisely as shown and described.

What is claimed is:

1. An implantable intragastric volume occupying system for the treatment of obesity comprising:
a volume occupying device configured to occupy volume within a patient's stomach;
an implantable sensor coupled to the volume occupying device and configured to sense a biological characteristic of the patient; and
an external control device configured to receive a telemetric signal sent in response to the biological characteristic being sensed by the implantable sensor, and to produce a notification to perform an action effective to vary the biological characteristic sensed by the implantable sensor.

2. The system of claim 1, wherein the biological characteristic sensed by the implantable sensor is a hormone level of the patient.

3. The system of claim 1, wherein the telemetric signal is sent telemetrically from a transmitter integrated with the implantable sensor.

4. The system of claim 1, wherein the notification is selected from a group consisting of a visual notification, an auditory notification, a movement of the external control device, and combinations thereof.

5. The system of claim 1, wherein the action is selected from a group consisting of injection of a hormone into the patient's body; inhalation of a hormone by the patient; drinking of a hormone by the patient; application of a patch to the patient's body being capable of distributing a hormone to the patient; spraying of a hormone into the patient's mouth; swallowing of a pill by the patient containing a hormone; insertion of a gum or film containing a hormone into the patient's mouth; and combinations thereof.

6. An implantable intragastric volume occupying device system for the treatment of obesity comprising:
a volume occupying device configured to occupy volume within a patient's stomach, and including a reservoir configured to contain an active agent being effective, when released into the patient, to at least assist in effecting weight loss in the patient, at least a portion of the volume occupying device including a semi-permeable membrane configured to allow the active agent to exit the reservoir and contact a portion of the patient's body.

7. The system of claim 6, wherein the volume occupying device includes an inflatable balloon.

8. The system of claim 7, wherein the inflatable balloon has a shape selected from a group consisting of a substantially spherical shape, a substantially disk-like shape, and combinations thereof.

9. The system of claim 7, wherein the reservoir is positioned within the inflatable balloon.

10. The system of claim 6, wherein at least a portion of the volume occupying device forms a shell around the reservoir.

11. The system of claim 6, wherein the volume occupying device includes a tube extending from the reservoir, and the semi-permeable membrane forms at least a portion of a tube.

12. The system of claim 6, wherein the volume occupying device includes a tube having a first end connected to the reservoir and a second end configured to dispense the active agent from the reservoir to a portion of the patient's body.

13. The system of claim 12, wherein the second end of the tube is configured to be positioned in the patient's upper intestine and dispense the active agent from the reservoir to the patient's upper intestine.

14. The system of claim 6, wherein the semi-permeable membrane forms an outer surface of the reservoir.

15. The system of claim 6, further comprising a pump configured to allow the active agent to exit the reservoir and contact a portion of the patient's body.

16. The system of claim 15, further comprising a sensor configured to detect a hormone level of the patient.

17. The system of claim 16, wherein the sensor is configured to transmit a signal to the pump in response to a hormone level detected by the sensor.

18. The system of claim 6, wherein the reservoir includes a one-way valve.

19. The system of claim 18, wherein the one-way valve is configured to allow the active agent to exit the reservoir in response to a force exerted against the reservoir by the patient's stomach or by food in the patient's stomach.

20. The system of claim 6, further comprising an electrode configured to produce an electrical charge on a side of the semi-permeable membrane, to enhance or impede diffusion of the active agent through the semi-permeable membrane.

21. The system of claim 6, further comprising an electrode configured to apply a voltage to the semi-permeable membrane.

22. The system of claim 21, wherein the voltage causes a size of a pore of the semi-permeable membrane to vary.

23. The system of claim 6, wherein the semi-permeable membrane is made from a material having a property that causes a size of a pore of the semi-permeable membrane to vary automatically in response to an environmental condition in the patient's body.

24. The system of claim 6, wherein the reservoir is configured to store bacteria that produce the active agent.

25. The system of claim 24, wherein pores of the semi-permeable membrane are sized to prevent the bacteria from exiting the reservoir.

26. The system of claim 6, further comprising an active agent.

27. The system of claim 26, wherein the active agent is selected from a group consisting of Glucagon-like peptide (GLP-1), Oxyntomodulin (OXM), Peptide YY (PYY), Pancreatic Polypeptide (PP), Insulin, Leptin, Gastrin, Gherlin blocker, inhibitors of DPP-IV, Amylin, Cholecystokinin (CCK), Pro-opiomelanocortin (POMC), and combinations thereof.

28. The system of claim 6, further comprising an electrode coupled to the volume occupying device and being configured to apply electric stimulation to a portion of the patient's body.

29. The system of claim 6, wherein an outer surface of the volume occupying device is coated with an active agent being effective, when released into the patient, to at least assist in effecting weight loss in the patient.

30. A method for the treatment of obesity comprising the step of:
implanting a volume occupying device into a patient's stomach, the volume occupying device being configured to occupy volume within the patient's stomach, and including a reservoir configured to contain an active agent being effective, when released into the patient, to at least assist in effecting weight loss in the patient, at least a portion of the volume occupying device including a semi-permeable membrane configured to allow the active agent to exit the reservoir and contact a portion of the patient's body.

31. The method of claim 30, wherein the volume occupying device includes an inflatable balloon.

32. The method of claim 31, further comprising a step of inflating the inflatable balloon.

33. The method of claim 32, wherein the active agent fills the inflatable balloon to inflate the inflatable balloon.

34. The method of claim 30, wherein the volume occupying device includes a tube having a first end coupled to the reservoir and a second end configured to dispense the active agent from the reservoir to the portion of the patient's body.

35. The method of claim 34, further comprising a step of positioning the second end of the tube in the patient's upper intestine.

36. A method for the treatment of obesity comprising the steps of:
inserting an electrode into a patient's body laparoscopically; and
coupling the electrode to the lower third of the patient's esophagus, the electrode configured to apply electric stimulation to the lower third of the patient's esophagus,
wherein the electrode is utilized in combination with a volume occupying device positioned within the patient's stomach.

37. The method of claim 36, wherein the electric stimulation promotes a sensation of satiety for the patient.

38. The method of claim 36, wherein the electrode is coupled to a control device configured to cause the electrode to apply the electric stimulation to the lower third of the patient's esophagus.

39. The method of claim 38, wherein the volume occupying device is configured to transmit a signal to the control device that causes the electrode to apply the electric stimulation to the lower third of the patient's esophagus.

40. The method of claim 39, wherein the volume occupying device is configured to transmit the signal in response to a force being exerted against the volume occupying device by the patient's stomach or by food in the patient's stomach.

41. The method of claim 36, wherein the step of inserting the electrode into the patient's body laparoscopically further comprises inserting a plurality of electrodes into the patient's body laparoscopically, and the step of coupling the electrode to the lower third of the patient's stomach further comprises coupling the plurality of electrodes to the lower third of the patient's esophagus along the vagus nerve.
